(19) United States
(12) Patent Application Publication
(10) Pub. No.: US 2014/0018676 A1
(43) Pub. Date: Jan. 16, 2014

(54) METHOD OF GENERATING TEMPERATURE MAP SHOWING TEMPERATURE CHANGE AT PREDETERMINED PART OF ORGAN BY IRRADIATING ULTRASOUND WAVE ON MOVING ORGANS, AND ULTRASOUND SYSTEM USING THE SAME

(71) Applicant: SAMSUNG ELECTRONICS CO., LTD., SUWON-SI (KR)

(72) Inventors: DONG-GEON KONG, YONGIN-SI (KR); KI-WAN CHOI, ANYANG-SI (KR); JI-YOUNG PARK, YONGIN-SI (KR); WON-CHUL BANG, SEONGNAM-SI (KR)

(73) Assignee: SAMSUNG ELECTRONICS CO., LTD., SUWON-SI (KR)

(21) Appl. No.: 13/777,187

(22) Filed: Feb. 26, 2013

(30) Foreign Application Priority Data
Jul. 11, 2012 (KR) 10-2012-0075747

(51) Int. Cl. A61B 5/01; A61B 8/00; A61N 7/02; A61B 5/00; A61B 8/08; A61B 8/14

(52) U.S. Cl. CPC A61B 5/015; A61B 8/5223; A61B 8/14; A61N 7/02; A61B 5/4836; A61B 8/543; A61B 8/466; A61B 8/08
USPC 600/438

(57) ABSTRACT

A method of generating a temperature map showing a temperature change in a predetermined part by irradiating ultrasound waves on a moving organ includes generating reference frames indicating images of an observed part including a treatment part in the predetermined organ during a predetermined period related to a movement cycle of the predetermined organ from echo signals transduced from reflected waves of ultrasound waves for diagnosis irradiated on the observed part during the predetermined period; generating a current frame indicating an image of the observed part at a time an ultrasound wave for treatment is irradiated on the treatment part from the echo signals; selecting a comparison frame that is one of the reference frames based on a similarity between the reference frames and the current frame; and generating a temperature map showing a temperature change in the observed part based on a difference between the comparison and current frames.

### CROSS-REFERENCE TO RELATED APPLICATIONS

This application claims the priority benefit of Korean Patent Application No. 10-2012-0075747, filed on Jul. 11, 2012, in the Korean Intellectual Property Office, the disclosures of which are incorporated herein in their entirety by reference.

### BACKGROUND

1. Field

The following description relates to a method of generating a temperature map showing a temperature change at a predetermined part of an organ by irradiating an ultrasound wave on moving organs, and an apparatus for generating a temperature map.

2. Description of the Related Art

Along with the development of medical science, a typical treatment for a tumor has developed from invasive surgeries, such as an abdominal operation, to minimally invasive surgeries. At present, non-invasive surgeries are also developed, and thus, a gamma knife, a cyber knife, a High Intensity Focused Ultrasound (HIFU) knife, and so forth are used. Particularly, among these knives, the recently commonly used HIFU knife is widely used in a therapy that is harmless to a human body and is eco-friendly by using ultrasound waves.

HIFU therapy using an HIFU knife is a surgery method for removing and curing a tumor by focusing and irradiating HIFU on a tumor part to be cured to cause focal destruction or necrosis of tumor tissue.

### SUMMARY

Provided are methods and apparatuses for a method of generating a temperature map showing a temperature change at a predetermined part of an organ by irradiating an ultrasound wave on moving organs, and an apparatus for generating a temperature map.

Additional aspects will be set forth in part in the description which follows and, in part, will be apparent from the description, or may be learned by practice of the presented embodiments.

According to an aspect of the present disclosure, a method of generating a temperature map showing a temperature change before and after an ultrasound wave for treatment is irradiated on a treatment part of a predetermined organ includes generating a plurality of reference frames indicating images of an observed part including a treatment part in the predetermined organ in a patient during a predetermined period related to a movement cycle of the predetermined organ from echo signals that are transduced from reflected waves of ultrasound waves for diagnosis irradiated on the observed part during the predetermined period; generating a current frame indicating an image of the observed part at a time an ultrasound wave for treatment is irradiated on the treatment part from the echo signals that are transduced from the reflected waves of the ultrasound waves for diagnosis irradiated on the observed part; selecting a comparison frame that is one of the reference frames based on a similarity between the reference frames and the current frame; and generating a temperature map showing a temperature change in the observed part based on a difference between the comparison frame and the current frame.

The selecting of the comparison frame may include selecting a frame that is the most similar to the current frame from among the reference frames as the comparison frame.

The selecting of the comparison frame may include determining a frame that is the most similar to the current frame from among the reference frames based on a difference between pixel values of each of the reference frames and pixel values of the current frame and selecting the reference frame, which is determined as the most similar frame to the current frame, as the comparison frame.

The predetermined period may include a breathing cycle of the patient that corresponds to the movement cycle of the predetermined organ, and the generating of the plurality of reference frames may include generating the reference frames during the breathing cycle of the patient.

The predetermined period may include a pause period between a breathing motion in which the movement of the predetermined organ is relatively small in the movement cycle of the predetermined organ, and the generating of the plurality of reference frames may include generating the reference frames during the pause period between the breathing motion.

The generating of the current frame may include generating the current frame from the echo signals that are transduced from the reflected waves of the ultrasound waves for diagnosis irradiated on the observed part during the pause period between the breathing motion.

The generating of the current frame may include generating current frames indicating images of the predetermined organ from the echo signals that are transduced from the reflected waves of the ultrasound waves for diagnosis irradiated on a plurality of cross-sectional images forming the observed part, and the generating of the temperature map may include generating a three-dimensional (3D) temperature map by accumulating a plurality of temperature maps generated from the generated current frames.

The selecting of the comparison frame may include selecting candidate reference frames from among the plurality of reference frames by considering an estimated position of the observed part at a time corresponding to the movement cycle of the predetermined organ or a time the current frame is generated.

Each of the reference frames may be obtained by replacing a reference frame generated at a time corresponding to a time the current frame is generated with the current frame by considering the movement cycle of the predetermined organ.

Each of the generating of the temperature map may include generating the temperature map by detecting a different type of waveform change between echo signals for generating the comparison frame selected from among the reference frames and echo signals for generating the current frame.

According to an aspect of the present disclosure, an ultrasound system to generate a temperature map showing a temperature change before and after an ultrasound wave for treatment is irradiated on a treatment part of a predetermined organ in a patient may include: an ultrasound diagnosis device to irradiate ultrasound waves for diagnosis on an observed part including the treatment part in the predetermined organ inside the patient during a predetermined period related to a movement cycle of the predetermined organ; an ultrasound treatment device to irradiate the ultrasound waves for treatment on the treatment part; and an ultrasound data processing device to generate the temperature map showing the temperature change in the observed part based on a difference between any one of a plurality of reference frames indicating images of the observed part that are generated from echo signals transduced from reflected waves of the ultrasound waves for diagnosis irradiated during the predetermined period and a current frame indicating an image of the observed part that is generated at a time the ultrasound wave for treatment is irradiated on the treatment part from the echo signals that are transduced from the reflected waves of the ultrasound waves for diagnosis.

The ultrasound data processing device may include a comparison frame generator for selecting a frame that is the most similar to the current frame from among the reference frames as a comparison frame.

The comparison frame generator determines a frame that is the most similar to the current frame from among the reference frames based on a difference between pixel values of each of the reference frames and pixel values of the current frame and selects the reference frame, which is determined as the most similar frame to the current frame, as the comparison frame.

The predetermined period is a breathing cycle of the patient that corresponds to the movement cycle of the predetermined organ, and the ultrasound data processing device may include a reference frame generator for generating the reference frames during the breathing cycle of the patient.

The predetermined period may include a pause period between a breathing motion in which the movement of the predetermined organ is relatively small in the movement cycle of the predetermined organ, and the ultrasound data processing device may include a reference frame generator for generating the reference frames during the pause period between the breathing motion.

The reference frame generator generates the current frame from the echo signals that are transduced from the reflected waves of the ultrasound waves for diagnosis irradiated on the observed part during the pause period between the breathing motion.

The ultrasound data processing device may include: a current frame generator to generate current frames indicating images of the predetermined organ from the echo signals that are transduced from the reflected waves of the ultrasound waves for diagnosis irradiated on a plurality of cross-sectional images forming the observed part; and a temperature map generator to generate a three-dimensional (3D) temperature map by accumulating a plurality of temperature maps generated from the generated current frames.

The reference frame generator may include a reference frame selector to select candidate reference frames from among the plurality of reference frames by considering an estimated position of the observed part at a time corresponding to the movement cycle of the predetermined organ or a time the current frame is generated.

The reference frame generator may replace a reference frame generated at a time corresponding to a time the current frame is generated with the current frame by considering the movement cycle of the predetermined organ.

### BRIEF DESCRIPTION OF THE DRAWINGS

The patent or application file contains at least one drawing executed in color. Copies of this patent or patent application publication with color drawings will be provided by the Office upon request and payment of the necessary fee. These and/or other aspects will become apparent and more readily appreciated from the following description of the embodiments, taken in conjunction with the accompanying drawings in which:

FIG. 1A is a conceptual diagram of an ultrasound system according to an embodiment of the present disclosure;

FIG. 1B is a configuration diagram of an ultrasound treatment apparatus according to an embodiment of the present disclosure;

FIG. 2 is a block diagram of an ultrasound data processing device in the ultrasound system of FIG. 1A, according to an embodiment of the present disclosure;

FIG. 3 is a block diagram of a reference frame generator in the ultrasound data processing device of FIG. 2, according to an embodiment of the present disclosure;

FIGS. 4A to 4E are diagrams for describing an operation of the reference frame generator of FIG. 3, according to an embodiment of the present disclosure;

FIGS. 5A to 5C are diagrams for describing an operation of a comparison frame selector shown in FIG. 3, according to an embodiment of the present disclosure;

FIG. 6 is a graph showing a measured movement displacement of a predetermined internal organ, according to an embodiment of the present disclosure;

FIGS. 7A and 7B are images for describing operations of a comparator and a temperature map generator in the ultrasound data processing device of FIG. 2, according to an embodiment of the present disclosure;

FIG. 8 is a flowchart illustrating a method of generating a temperature map of an organ using an ultrasound wave, according to an embodiment of the present disclosure;

FIGS. 9A to 9H are diagrams for describing a method of generating, by a controller, an image suitable for rapid and accurate tracking of a predetermined internal organ including a treatment part from medical images of a patient for a predetermined period, according to an embodiment of the present disclosure;

FIG. 10 is a flowchart illustrating a method of generating a temperature map of a moving organ using an ultrasound wave in an ultrasound treatment and diagnosis system for treating a patient in response to the movement of an internal organ, according to an embodiment of the present disclosure;

FIG. 11 is a diagram for describing constructing a reference frame database (DB) in the reference frame generator (operation 1050) in the method of FIG. 10, according to an embodiment of the present disclosure;

FIG. 12 is a diagram for describing a pause between a breathing motion;

FIG. 13 is a flowchart illustrating a method of measuring a temperature of an internal organ using an ultrasound wave in an ultrasound treatment and diagnosis system for treating the internal organ in a pause between a breathing motion, according to an embodiment of the present disclosure;

FIG. 14 is a diagram for describing constructing a reference frame DB in the reference frame generator (operation 1350) in the method of FIG. 13, according to an embodiment of the present disclosure; and

FIG. 15 is a diagram for describing a method of generating a temperature map that is characterized in that an ultrasound diagnosis device operates at a fixed position thereof in an ultrasound treatment and diagnosis system for treating an internal organ, according to an embodiment of the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

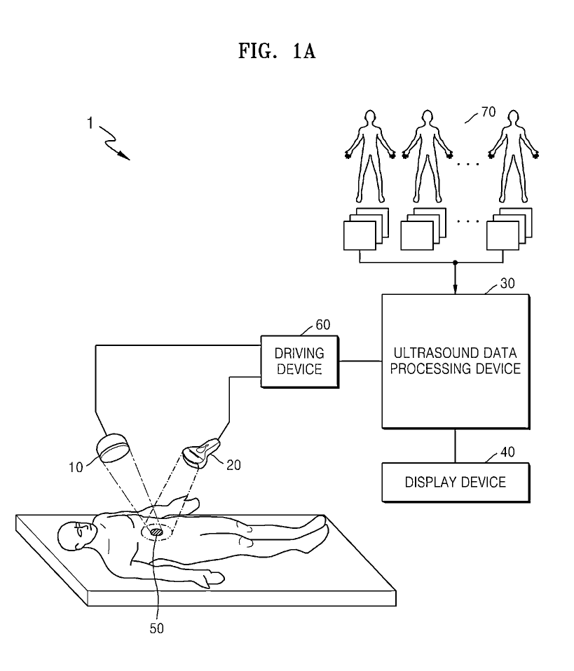

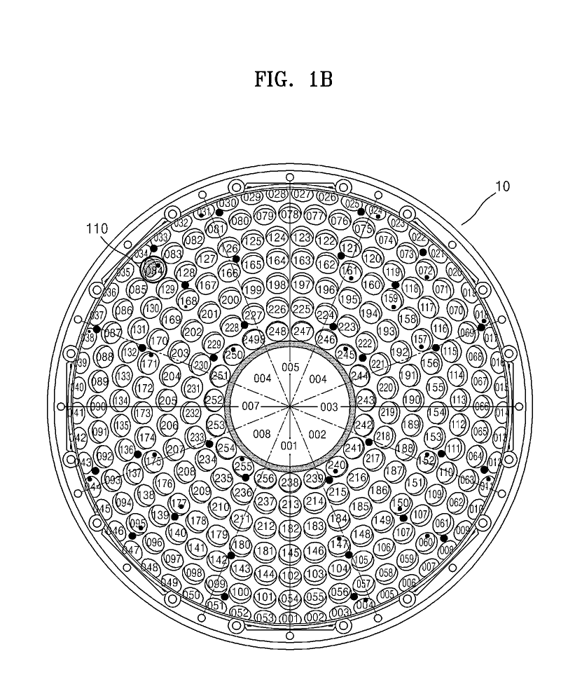

FIG. 1A is a conceptual diagram of an ultrasound system 1 according to an embodiment of the present disclosure. Referring to FIG. 1A, the ultrasound system 1 includes an ultrasound treatment device 10, an ultrasound diagnosis device 20, an ultrasound data processing device 30, a display device 40, and a driving device 60. Only components associated with the current embodiment are included in the ultrasound system 1 shown in FIG. 1A. Thus, it will be understood by one of ordinary skill in the art that other general-use components may be further included in addition to the components shown in FIG. 1A. In addition, external medical images captured by medical experts for the diagnosis of patients may be input to the ultrasound data processing device 30, according to an embodiment of the present disclosure to be described below.

When a tumor in a patient is treated, the ultrasound treatment device 10 in the ultrasound system 1 heats the tumor by irradiating an ultrasound wave for treatment on a treatment part 50 of the tumor, and the ultrasound diagnosis device 20 irradiates an ultrasound wave for diagnosis on a surrounding part (hereinafter, referred to as “observed part”) including the treatment part 50 and receives reflected waves of the irradiated ultrasound wave. Thereafter, the ultrasound system 1 transduces the received reflected waves to echo signals, acquires ultrasound images based on the echo signals, and diagnoses whether a therapy has been completed. The heat indicates focal destruction or necrosis of tissue in the treatment part 50. In detail, the ultrasound system 1 treats the treatment part 50 using the ultrasound treatment device 10 for irradiating the ultrasound wave for treatment on the treatment part 50, e.g., a portion of the tumor, in the body of the patient and monitors treatment results, such as a temperature of the treatment part 50, using the ultrasound diagnosis device 20 for irradiating the ultrasound wave for diagnosis on the observed part.

The ultrasound treatment device 10 may be called a treatment probe. The ultrasound treatment device 10 may irradiate the ultrasound wave for treatment on various parts of a patient while moving under control of the driving device 60. Alternatively, the ultrasound treatment device 10 may irradiate the ultrasound wave for treatment on various parts of a patient in a method of changing a focal position at which the ultrasound wave for treatment is irradiated at a fixed position thereof. That is, the ultrasound treatment device 10 generates the ultrasound wave for treatment and irradiates the ultrasound wave for treatment on local tissue of a patient. As the ultrasound wave for treatment, High Intensity Focused Ultrasound (HIFU) having enough energy for necrosis of a tumor in the body of a patient may be used. That is, the ultrasound treatment device 10 corresponds to a device for irradiating HIFU generally known as the ultrasound wave for treatment. Because the HIFU is well-known to one of ordinary skill in the art, a detailed description thereof is omitted. However, it will be understood by one of ordinary skill in the art that the ultrasound treatment device 10 is not limited to the device for irradiating HIFU and any device may be included in the scope of the ultrasound treatment device 10 as long as similarly to the device for irradiating HIFU.

The method of changing a focal position at which the ultrasound wave for treatment is irradiated at a fixed position of the ultrasound treatment device 10 may use a Phase Array (PA) method. The PA method uses the premise that the ultrasound treatment device 10 includes a plurality of elements 110, as shown in FIG. 1B, wherein the plurality of elements 110 may individually irradiate an ultrasound wave upon receiving a signal from the driving device 60 and may have differently set timings for irradiating the ultrasound waves. The individual irradiation of an ultrasound wave by the plurality of elements 110 may enable the ultrasound treatment device 10 to irradiate along with a moving lesion at a fixed position of the ultrasound treatment device 10. Thus, the PA method has the same effect as a method of irradiating an ultrasound wave while the ultrasound treatment device 10 is physically moving. Because the PA method is well-known to one of ordinary skill in the art, a detailed description thereof is omitted. In addition, although the ultrasound treatment device 10 is formed in a circular shape in FIG. 1B, the ultrasound treatment device 10 may be formed in various shapes, such as a rectangle, only if the ultrasound treatment device 10 is represented by a sum of the plurality of elements 110.

The ultrasound diagnosis device 20 may be called a diagnosis probe. The ultrasound diagnosis device 20 irradiates the ultrasound wave for diagnosis towards the observed part under control of the driving device 60. The observed part may be wider than or the same as the treatment part 50. In addition, the ultrasound diagnosis device 20 receives reflected waves of the irradiated ultrasound wave for diagnosis from the part on which the ultrasound wave for diagnosis is irradiated. In detail, the ultrasound diagnosis device 20 is generally produced with a piezoelectric transducer. When an ultrasound wave in a range from approximately 2 MHz to approximately 18 MHz is propagated to a predetermined part in the body of a patient from the ultrasound diagnosis device 20, the ultrasound wave is partially reflected from layers between several different tissues. In particular, the ultrasound wave is reflected from places in the body in which density changes, e.g., blood cells in blood plasma, small tissue in organs, etc. These reflected ultrasound waves, i.e., the reflected waves, cause the piezoelectric transducer to vibrate and output electrical pulses in response to the vibration. In the current embodiment, echo signals transduced from reflected waves received by the ultrasound diagnosis device 20 are additionally used to monitor a temperature change at the observed part. That is, the echo signals may be used to monitor a temperature change at the observed part in addition to generally known generation of an ultrasound diagnosis image. A method of monitoring a temperature change at the observed part will be described below. The ultrasound diagnosis device 20 may also be implemented at a fixed position thereof, and may be configured to have a size capable of accommodating a predetermined internal organ including the treatment part 50. An embodiment in a case where a position of the ultrasound diagnosis device 20 is fixed will be described below.

Although the ultrasound treatment device 10 and the ultrasound diagnosis device 20 are described as independent devices in the current embodiment, the current embodiment is not limited thereto, and the ultrasound treatment device 10 and the ultrasound diagnosis device 20 may be implemented as individual modules in a single device or implemented as a single device. That is, the ultrasound treatment device 10 and the ultrasound diagnosis device 20 are not limited to only a certain form. In addition, the ultrasound treatment device 10 and the ultrasound diagnosis device 20 are not limited to being singular, and may each be plural. In addition, although the ultrasound treatment device 10 and the ultrasound diagnosis device 20 irradiate ultrasound waves downwards above the body of a patient in FIG. 1A, a method of irradiating ultrasound waves in various directions, e.g., a method of irradiating ultrasound waves upwards, from below the body of a patient, may be implemented.

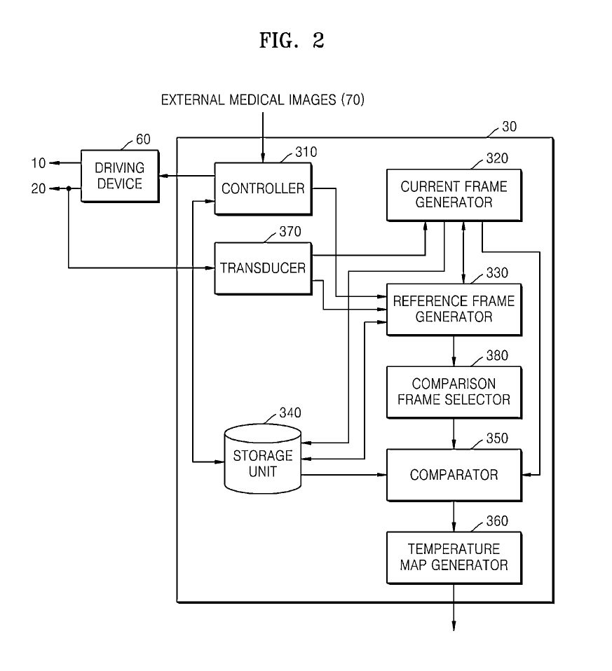

The driving device 60 controls positions of the ultrasound treatment device 10 and the ultrasound diagnosis device 20. In detail, the driving device 60 receives position information of the treatment part 50 from a controller (310 of FIG. 2) to be described below and controls a position of the ultrasound treatment device 10 so that the ultrasound treatment device 10 correctly irradiates the ultrasound wave for the treatment on the treatment part 50, and receives position information of the observed part from the controller (310 of FIG. 2) to be described below and controls a position of the ultrasound diagnosis device 20 so that the ultrasound diagnosis device 20 correctly irradiates the ultrasound wave for the diagnosis on the observed part and receives reflected waves of the ultrasound wave for the diagnosis. When the ultrasound treatment device 10 is used in the PA method, the controller (310 of FIG. 2) to be described below measures the displacement of a moving organ in response to a breathing motion and calculates a timing when each element 110 forming the ultrasound treatment device 10 irradiates an ultrasound wave in response to the movement of the treatment part 50 in the organ. Thereafter, the controller 310 transmits the calculated timing information to the driving device 60, and the driving device 60 transmits a command for irradiating the ultrasound wave for the treatment to each element 110 forming the ultrasound treatment device 10 in response to the received timing information.

As described above, the ultrasound system 1 also monitors a temperature change at the observed part using the ultrasound diagnosis device 20. In a case of an ultrasound therapy using the ultrasound wave for the treatment, such as the HIFU, when the HIFU arrives at a portion of a tumor, a temperature of this tumor portion may instantaneously increase to more than 70° C. due to heat energy caused by the HIFU. Theoretically, it is known that tissue destruction occurs within approximately 110 msec at a temperature of approximately 60° C. This high temperature causes coagulative necrosis of tissue and blood vessels in the tumor portion. According to the current embodiment, by real-time monitoring of a temperature change at the observed part, it may be correctly perceived whether a therapy is to be continued or has been completed, so that an ultrasound therapy may be efficiently performed. In more detail, even when an internal organ moves due to breathing or other causes, a temperature change at the observed part may be monitored in real-time, and thus, it may be correctly perceived whether the ultrasound wave for treatment has been correctly irradiated on the treatment part 50 or whether a therapy is to be continued or has been completed.

FIG. 2 is a block diagram of the ultrasound data processing device 30 in the ultrasound system 1 of FIG. 1A, according to an embodiment of the present disclosure. Referring to FIG. 2 the ultrasound data processing device 30 includes the controller 310, a current frame generator 320, a reference frame generator 330, a storage unit 340, a comparator 350, a temperature map generator 360, a transducer 370, and a comparison frame selector 380. For ease of description, only components associated with the current embodiment are included in the ultrasound data processing device 30 shown FIG. 2. However, it will be understood by one of ordinary skill in the art that other general-use components may be further included in addition to the components shown in FIG. 2.

The controller 310 transmits position control signals indicating positions of the ultrasound treatment device 10 and the ultrasound diagnosis device 20 that are generated based on motion information of a predetermined organ in the body of a patient to the driving device 60. In detail, the controller 310 generates a position control signal with respect to a position at which the ultrasound treatment device 10 irradiates the ultrasound wave for treatment in response to the movement of the treatment part 50 in the organ by using displacement information measured based on the movement of the organ in response to a breathing motion and transmits the position control signal to the driving device 60. A process of acquiring movement information of a predetermined organ in the body of a patient is a preparation process for a medical expert to diagnose a patient and may be performed even outside of an operating room. For example, a movement displacement of a liver due to breathing is as shown in FIG. 6. In the movement displacement graph of FIG. 6, a period 610 showing a relatively large movement magnitude indicates an inhalation or exhalation period of a breath, and a period 620 showing a relatively small movement magnitude indicates a pause period between a breathing motion. These inhalation, exhalation, and pause periods of a breathing motion are periodically repeated.

In addition, the controller 310 generates a position control signal with respect to a position at which the ultrasound diagnosis device 20 irradiates the ultrasound wave for diagnosis and receives reflected waves thereof and transmits the position control signal to the driving device 60. The controller 310 may generate a position control signal for the ultrasound diagnosis device 20 so that the ultrasound diagnosis device 20 periodically irradiates the ultrasound wave for diagnosis on every section equal to or less than 0.2 mm on the observed part, to obtain a plurality of reference frames to be described below. For example, the controller 310 may generate an image suitable for rapid and accurate tracking of a predetermined internal organ including the treatment part 50 from medical images for a breathing cycle of a patient to generate position control signals for the ultrasound treatment device 10 and the ultrasound diagnosis device 20, and an embodiment of this method will be described below.

The transducer 370 receives, from the ultrasound diagnosis device 20, reflected waves of the ultrasound wave for diagnosis that are received by the ultrasound diagnosis device 20. Thereafter, the transducer 370 transduces the reflected waves of the ultrasound wave for diagnosis into echo signals. An echo signal indicates a received beam formed an ultrasound Radio Frequency (RF) signal or a signal from which anatomic information of a medium, such as a B-mode image, is identified and temperature-related parameters are extracted through processing. Thereafter, the transducer 370 transmits the echo signals to the current frame generator 320 and the reference frame generator 330 to be described below.

The current frame generator 320 receives echo signals that are transduced from reflected waves of the ultrasound wave for diagnosis that are irradiated on the observed part by the ultrasound diagnosis device 20 at a current time, i.e., when the ultrasound treatment device 10 irradiates the ultrasound wave for treatment on the treatment part 50, and generates a current frame indicating an image of the observed part at the current time based on the received echo signals. The current frame includes information about a position and temperature of the observed part. An example of displaying the current frame with different brightness values may be a B-mode image. The B-mode image indicates an image in which echo signals transduced from reflected waves of the ultrasound wave for diagnosis are expressed by brightness differences. In detail, a brightness value in a B-mode image may increase in correspondence with the magnitude of an echo signal. The current frame generator 320 may determine whether the current frame generated by the current frame generator 320 is a current frame generated in a pause period between a breathing motion. The pause period between a breathing motion indicates a period in which a movement magnitude of an organ is relatively smaller than an inhalation or exhalation period within one breathing cycle.

The operation described above indicates a case where the current frame generator 320 generates a single current frame. However, the current frame generator 320 may generate a plurality of current frames. That is, for the temperature map generator 360, to be described below, to generate a completed temperature map of a three-dimensional (3D) volume for the observed part, the ultrasound diagnosis device 20 may receive reflected waves of ultrasound waves for diagnosis that are irradiated while changing a position and orientation thereof, and the current frame generator 320 may generate a plurality of current frames indicating a plurality of cross-sectional images forming the observed part by using echo signals transduced from the reflected waves.

The reference frame generator 330 receives echo signals transduced from reflected waves of ultrasound waves for diagnosis from the transducer 370 and generates reference frames indicating an image of the observed part at a corresponding time by using the received echo signals. Each of the reference frames includes information about a position and temperature of the observed part. The observed part may specify a proper part including the treatment part 50 in a predetermined internal organ. Each of the reference frames is generally generated as a frame including temperature information of the observed part before the ultrasound wave for treatment is irradiated on the treatment part 50 by the ultrasound treatment device 10. That is, to finally observe a relative temperature change between before and after the ultrasound wave for treatment is irradiated on the treatment part 50, the reference frames may be generated before the ultrasound wave for treatment is irradiated on the treatment part 50 by the ultrasound treatment device 10.

Alternatively, a current frame generated when the ultrasound treatment device 10 irradiates the ultrasound wave for treatment on the treatment part 50 may be used as a reference frame. This is implemented by a method of updating a reference frame database (DB) by a current frame, which is described below. This causes a reference frame to be generated in a process of irradiating the ultrasound wave for treatment on the treatment part 50 in the ultrasound treatment device 10 instead of generating the reference frame before the ultrasound treatment device 10 irradiates the ultrasound wave for treatment on the treatment part 50. The reference frame DB updated by the current frame is used when temperature-related parameters are extracted by Echo-Shift (ES) method. The ES method is described below. A detailed description of the method of updating the reference frame DB will be made below.

The storage unit 340 stores the current frame generated by the current frame generator 320 or the reference frames generated by the reference frame generator 330, respectively.

The comparator 350 generates a temperature map of the current frame by comparing the echo signals forming the current frame generated by the current frame generator 320 with the echo signals forming the comparison frame selected by the comparison frame selector 380 so that the temperature map generator 360 generates a completed temperature map from which a temperature change of the observed part is observed according to various criteria, and this is implemented by extracting temperature-related parameters. The comparator 350 generates a temperature map of the current frame that corresponds to a temperature change between the observed part shown in a reference frame and the observed part shown in the current frame based on a result of extracting the temperature-related parameters. For example, the temperature map of the current frame indicates a map displaying a physical amount proportional to a temperature, a map displaying a relative temperature change between the observed part shown in a reference frame and the observed part shown in the current frame, or a map displaying an unconditional temperature of the observed part shown in the current frame, etc.

A method of generating the map displaying a relative temperature change between the observed part shown in a reference frame and the observed part shown in a current frame will now be described. As a method of extracting temperature-related parameters, a Change in Backscattered Energy (CBE) method, the ES method, and a method of calculating a change of B/A are known.

A method of extracting temperature-related parameters using the CBE method is first described. The comparator 350 compares echo signals forming a reference frame with echo signals forming a current frame and detects an amplitude-changed portion from the echo signals forming the current frame. Thereafter, the comparator 350 detects a temperature change corresponding to a detected amplitude-changed level from a mapping table stored in the storage unit 340 and generates a temperature map of the current frame that corresponds to a relative temperature change between the observed part shown in the reference frame and the observed part shown in the current frame by using the detected temperature change value. The mapping table includes amplitude change values of a plurality of echo signals predefined as able to be transduced from reflected waves of the ultrasound wave for diagnosis and temperature change values mapped one-to-one to the amplitude change values. In the mapping table, a temperature change value mapped to a certain amplitude change value indicates a temperature change value of the treatment part 50 that is predicted from the certain amplitude change value. According to an embodiment of the present disclosure, a comparison frame selected from among reference frames generated before the ultrasound treatment device 10 irradiates the ultrasound wave for treatment on the treatment part 50 may be compared with a current frame generated when the ultrasound treatment device 10 irradiates the ultrasound wave for treatment on the treatment part 50.

Next, a method of extracting temperature-related parameters using the ES method is described. The comparator 350 compares echo signals forming a reference frame with echo signals forming a current frame, detects a portion in which an echo signal speed (i.e., echo time) is changed, i.e., a portion in which an echo signal delay occurs, from among the echo signals forming the current frame, and calculates a delay variation by differentiating the echo signal delay by a distance. Thereafter, the comparator 350 detects a temperature change corresponding to a detected echo signal delay variation level from a mapping table stored in the storage unit 340 and generates a temperature map of the current frame that corresponds to a relative temperature change between the observed part shown in the reference frame and the observed part shown in the current frame by using the detected temperature change value. The mapping table may be obtained by considering a speed change and thermal expansion in tissue according to a temperature. In the mapping table, a temperature change value mapped to a value of a certain echo signal delay variation level indicates a temperature change value of the treatment part 50 that is predicted from the value of the certain echo signal delay variation level. According to an embodiment of the present disclosure, a current frame generated when the ultrasound treatment device 10 irradiates the ultrasound wave for treatment on the treatment part 50 may be compared with a comparison frame, selected from among reference frames, generated at a time approximately equal to a time the current frame is generated. The reason is because the temperature map of the current frame corresponding to a relative temperature change between the observed part shown in the reference frame and the observed part shown in the current frame may show a large difference from an actual temperature change, if a time difference between when the comparison frame selected from among the reference frames is generated and when the current frame is generated is large in the ES method.

Finally, a method of extracting temperature-related parameters using the method of calculating a change of B/A is described. B/A denotes a value indicating a nonlinear characteristic of an echo signal speed changed in response to a temperature of the observed part on which the ultrasound wave for diagnosis is irradiated. B/A is described in detail in “Estimation of temperature distribution in biological tissue by acoustic nonlinearity parameter” (written by Zhang, D., Gong, X. F.) published in 2006. The comparator 350 compares B/A values of the echo signals forming the reference frame with B/A values of the echo signals forming the current frame to detect a portion in which a B/A value is changed from among the echo signals forming the current frame. Thereafter, the comparator 350 detects a temperature change corresponding to a detected echo signal B/A change value from a mapping table stored in the storage unit 340 and generates a temperature map of the current frame that corresponds to a relative temperature change between the observed part shown in the reference frame and the observed part shown in the current frame by using the detected temperature change value. The mapping table includes B/A change values of a plurality of echo signals predefined as capable of being generated by irradiation of the ultrasound wave for diagnosis and temperature change values mapped one-to-one to the B/A change values. In the mapping table, a temperature change value mapped to a B/A change value of a certain echo signal indicates a temperature change value of the treatment part 50 that is predicted from the B/A change value of the certain echo signal.

The map displaying an unconditional temperature of the observed part shown in the current frame indicates a map displaying a correct temperature of the observed part shown in the current frame. In general, before the ultrasound treatment device 10 irradiates the ultrasound wave for treatment, a temperature of the observed part corresponds to a normal temperature of a human body. Thus, the comparator 350 extracts the parameters related to the temperature and generates a map displaying an unconditional temperature value by adding a body temperature of a patient to a relative temperature increase value of the observed part shown in the current frame that is compared with the observed part shown in the reference frame by using the extracted temperature-related parameters. A detailed method of extracting the temperature-related parameters is the same as described in the method of generating a map displaying a relative temperature change.

In addition, the map displaying a physical amount proportional to a temperature indicates a temperature map generated directly using delay variations, amplitude change values, or B/A values between the echo signals forming the reference frame and the echo signals forming the current frame. In general, because these values are proportional to a temperature, information about a temperature change may be obtained even though the physical amount is displayed as it is.

The temperature map generator 360 generates a completed temperature map from which a temperature change of the observed part is observed according to various criteria, by using the temperature map of the current frame that is generated by the comparator 350. A method of generating the completed temperature map is described in detail below.

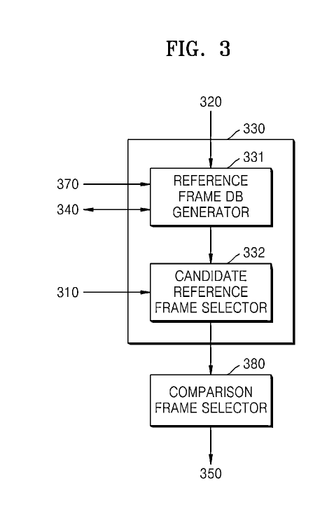

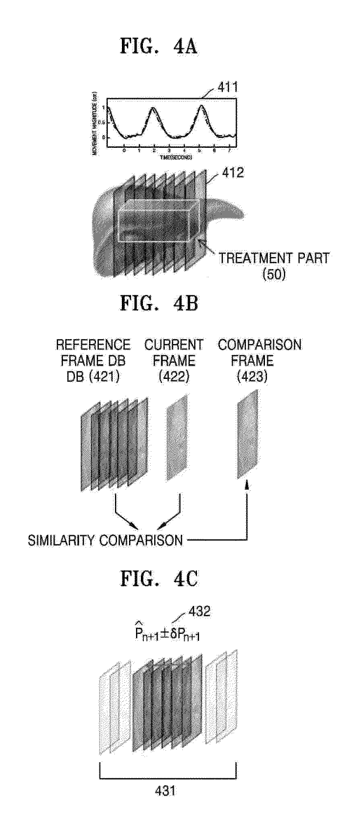

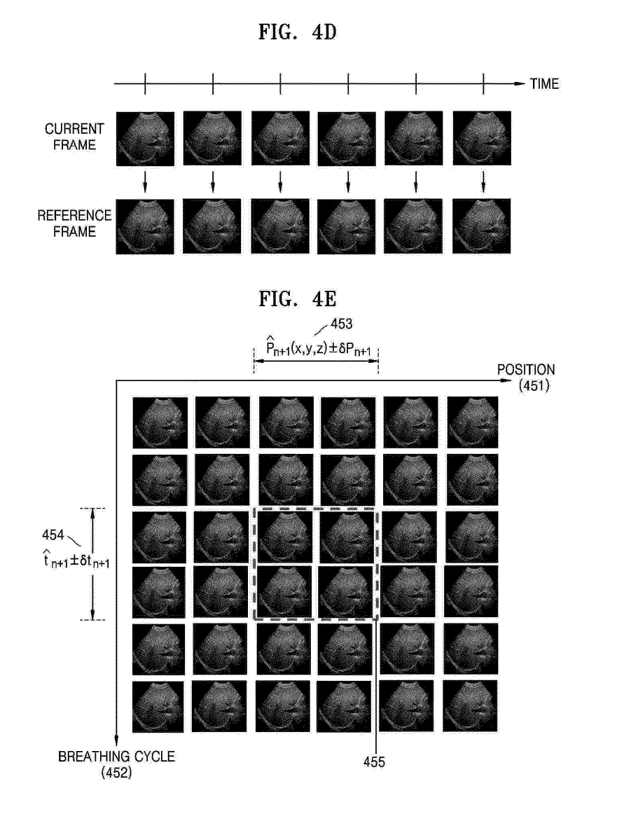

A comparison frame selecting process will now be described with reference to FIGS. 3, 4A, 4B, and 4C.

FIG. 3 is a block diagram of the reference frame generator 330 and the comparison frame selector 380, according to an embodiment of the present disclosure. The reference frame generator 330 may include a reference frame DB generator 331, and may further include a candidate reference frame selector 332, if necessary.

The reference frame DB generator 331 receives, from the transducer 370, echo signals that are transduced from reflected waves received by the ultrasound diagnosis device 20 and generates reference frames indicating an image of the observed part by using the received echo signals. In addition, the reference frame DB generator 331 receives, from the storage unit 340, reference frames that are previously generated by the reference frame DB generator 331 and stored in the storage unit 340 and builds a reference frame DB by gathering the reference frames generated by the reference frame DB generator 331 and the reference frames stored in the storage unit 340. In detail, as shown in FIG. 4A, the controller 310 measures a movement displacement of a predetermined internal organ including the treatment part 50 as shown in a graph 411 and transmits position information of the predetermined internal organ including the treatment part 50, which corresponds to the movement displacement, to the driving device 60. The movement displacement of the predetermined internal organ may indicate a movement displacement of the organ due to breathing of a human body. The driving device 60 receives position information transmitted from the controller 310 and controls a position of the ultrasound diagnosis device 20. The ultrasound diagnosis device 20 irradiates the ultrasound wave for diagnosis on the observed part, receives reflected waves thereof, and transmits the reflected waves to the transducer 370. The transducer 370 transduces the received reflected waves into echo signals and transmits the echo signals to the reference frame DB generator 331. The reference frame DB generator 331 generates reference frames indicating an image of the observed part by using the echo signals, as shown in an image 412. In addition, the reference frame DB generator 331 receives, from the storage unit 340, reference frames that are previously generated by the reference frame DB generator 331 and stored in the storage unit 340 and builds a reference frame DB 421 by gathering the reference frames generated by the reference frame DB generator 331 and the reference frames stored in the storage unit 340.

In addition, as shown in FIG. 4D, the reference frame DB generator 331 may receive a current frame generated by the current frame generator 320 from the current frame generator 320 and update reference frames. In detail, the reference frame DB generator 331 may update reference frames by replacing a reference frame generated at an arbitrary time in a breathing cycle before a current breathing cycle by a current frame generated at a corresponding time in the current breathing cycle. This causes a reference frame to be obtained while the ultrasound treatment device 20 is irradiating the ultrasound wave for treatment on the treatment part 50, and this obtained reference frame may be used to extract temperature-related parameters in the ES method.

An embodiment of an operation of the candidate reference frame selector 332 will now be described with reference to FIGS. 4C and 4E. The candidate reference frame selector 332 receives position information corresponding to a movement displacement of a predetermined internal organ from the controller 310 and selects candidate reference frames from a reference frame DB. In detail, the controller 310 generates position control signals for the ultrasound treatment device 10 and the ultrasound diagnosis device 20. In particular, the controller 310 may generate a position control signal for the ultrasound treatment device 10 to irradiate the ultrasound wave for treatment on the treatment part 50 along with the movement of an internal organ of a patient. An embodiment of generating a position control signal for the ultrasound treatment device 10 to irradiate along with the movement of an internal organ of a patient will be described below. In correspondence with that the controller 310 generates a position control signal for the ultrasound treatment device 10 so that the ultrasound treatment device 10 follows the movement of an internal organ of a patient, in detail, the movement of the treatment part 50, the controller 310 needs to generate a position control signal for the ultrasound diagnosis device 20 so that the ultrasound diagnosis device 20 also follows the movement of the observed part. As such, the ultrasound diagnosis device 20 receives reflected waves by irradiating the ultrasound wave for diagnosis on the observed part in response to a position control signal of the controller 310, and the current frame generator 320 generates a current frame by using echo signals transduced from the reflected waves. The candidate reference frame selector 332 selects a reference frame to be compared with such a generated current frame from among frames in the DB built by the reference frame DB generator 331. In detail, the candidate reference frame selector 332 may select candidate reference frames from among the frames in the DB built by the reference frame DB generator 331 and finally select a reference frame from the candidate reference frames.

A method of selecting candidate reference frames in the candidate reference frame selector 332 will now be described in detail. First, it is assumed that a current time a reference frame is generated in a breathing cycle of a human body is tₙ₊₁, and a time a previous reference frame is generated is tₙ. In addition, it is assumed that a central position of the treatment part 50 or the observed part at the time tₙ is Pₙ(x, y, z), and a central position thereof at the time tₙ₊₁ is Pₙ₊₁(x, y, z). In addition, the candidate reference frame selector 332 uses an error range ±δPₙ₊₁ of an estimated position of the observed part that is previously input by a user. That is, a movement displacement of a predetermined internal organ moving according to a breathing motion of a patient maintains a certain level of similarity, but a position of the predetermined internal organ may minutely vary. Thus, an estimated position of the observed part at an arbitrary time in a breathing cycle may be different from an actual position of the observed part at the arbitrary time. Accordingly, to select candidate reference frames, the candidate reference frame selector 332 may use \hat{P}ₙ₊₁(x, y, z) denoting an estimated position of the observed part and ±δPₙ₊₁ denoting an error range thereof. The estimated position \hat{P}ₙ₊₁(x, y, z) of the observed part is obtained from position information corresponding to a movement displacement of a predetermined internal organ which the candidate reference frame selector 332 receives from the controller 310, and the error range ±δPₙ₊₁ is pre-set as a predetermined proper error value by the user.

Thus, as shown in FIG. 4C, the candidate reference frame selector 332 may select reference frames in a range of \hat{P}ₙ₊₁±δPₙ₊₁ as candidate reference frames 432 from among reference frames 431 in a reference frame DB by using the estimated position of the observed part and the error range ±δPₙ₊₁ thereof that are described above. In addition, as shown in FIG. 4E, the candidate reference frame selector 332 may select reference frames in a common range of reference frames 453, which are generated at an estimated position 451 of the observed part and in an error range thereof, and reference frames 454, which are generated for a breathing cycle 452 of a patient and an error range thereof, as candidate reference frames 455 from among reference frames in a reference frame DB by considering both the estimated position 451 of the observed part and the breathing cycle 452 of the patient, which is measured by the controller 310.

As shown in FIG. 4B, the comparison frame selector 380 calculates a similarity between reference frames in the reference frame DB 421 and a current frame 422 generated by the current frame generator 320 and selects a reference frame having the highest similarity to the current frame 422 as a comparison frame 423. Alternatively, when the reference frame generator 330 includes the candidate reference frame selector 332, the comparison frame selector 380 calculates a similarity between reference frames 431 or 455 and the current frame 422 generated by the current frame generator 320 and selects a reference frame having the highest similarity to the current frame 422 as the comparison frame 423.

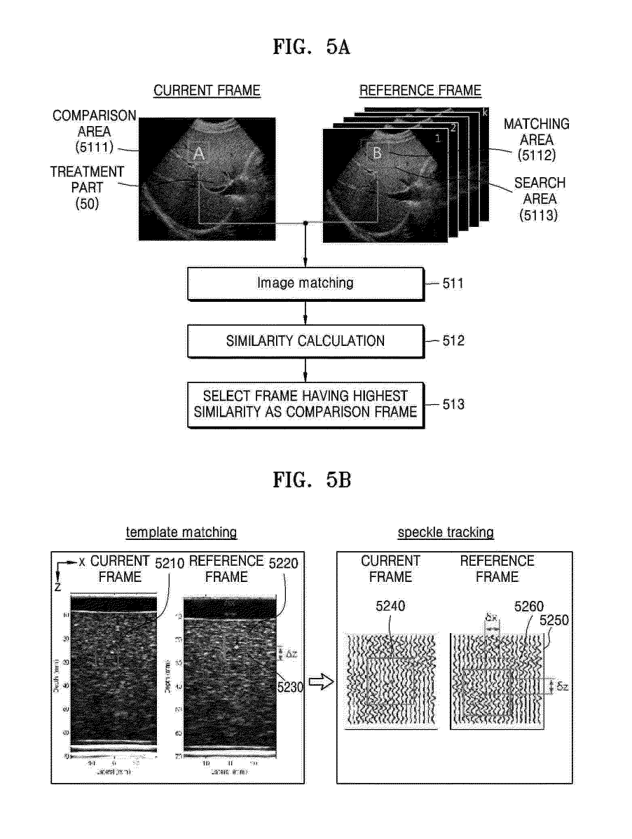

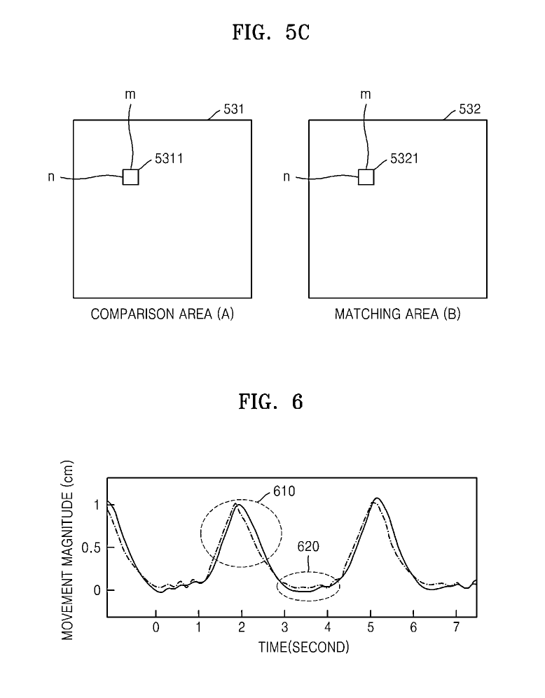

An embodiment of selecting, by the comparison frame selector 380, a comparison frame from among reference frames in a reference frame DB will now be described with reference to FIGS. 5A to 5C. As shown in FIG. 5A, the comparison frame selector 380 selects a comparison area 5111 from a current frame and performs image matching on a search area 5113 of each of the reference frames in operation 511 to find a matching area 5112 that is the most similar to the comparison area 5111. The search area 5113 indicates a position of each of the reference frames, which corresponds to a position at which the comparison area 5111 is located in the current frame. In addition, the search area 5113 is selected as a wider area including the comparison area 5111. Thereafter, the comparison frame selector 380 calculates a similarity between the matching area 5112 selected from each of the reference frames and the comparison area 5111 of the current frame in operation 512 and selects a reference frame having the most similarity as a comparison frame in operation 513. This process will now be described in detail.

First, the comparison frame selector 380 may specify the comparison area 5111 from the current frame. The comparison area 5111 may be selected by excluding an area on which the ultrasound treatment device 10 irradiates the ultrasound wave for treatment. The reason is because the treatment part 50 in the current frame that is the area on which the ultrasound treatment device 10 irradiates the ultrasound wave for treatment is not an area suitable to measure a similarity between a current frame and a reference frame before and after the ultrasound wave for treatment is irradiated, because ultrasound images before and after the ultrasound wave for treatment is irradiated may be different from each other due to tissue degeneration by energy of the ultrasound wave for treatment. Moreover, in addition to the exclusion of the treatment part 50 that is the area on which the ultrasound treatment device 10 irradiates the ultrasound wave for treatment, an area including many landmark points, such as blood vessels distributed in an internal organ, may be selected. The comparison area 5111 in the current frame may be selected in a singular or plural form.

Thereafter, the comparison frame selector 380 performs image matching between the search area 5113 and the comparison area 5111 in operation 511 to find the matching area 5112 that is an area most similar to the comparison area 5111. However, although a plurality of comparison areas 5111 may be selected from the current frame as described above, an embodiment of performing image matching in operation 511 when only one matching area 5112 is selected is described hereinafter. The image matching (operation 511) includes template matching and speckle matching. When a plurality of comparison areas 5111 are selected, the image matching (operation 511) to be described below is repeatedly performed for the plurality of comparison areas 5111.

The comparison frame selector 380 performs template matching between the comparison area 5111 in the current frame and the search area 5113 in the reference frame to find the matching area 5112 in the reference frame. The search area 5113 in the reference frame is selected as a wider area than the comparison area 5111 in the current frame. The comparison frame selector 380 performs the template matching to find the matching area 5112 in the pixel unit of an image. The comparison frame selector 380 performs the speckle tracking to determine the matching area 5112 more precisely than a pixel unit of an image.

Because concrete algorithms for performing the template matching and the speckle tracking are well-known to one of ordinary skill in the art, a detailed description thereof is omitted.

The left side of FIG. 5B shows an embodiment of performing the template matching between a current frame and a reference frame in the comparison frame selector 380. The comparison frame selector 380 selects a search area 5220 to be compared with a comparison area 5210 in the current frame from the reference frame. In detail, the comparison frame selector 380 performs the template matching in a method of performing the comparison by moving the comparison area 5210 pixel-by-pixel in the search area 5220 in the reference frame. The search area 5220 in the reference frame is selected as a wider area than the comparison area 5210 in the current frame. The template matching described above is a method of finding an area most similar to the comparison area 5210 in the current frame from the search area 5220 in the reference frame and has a precision of an image pixel unit in terms of resolution.

The comparison frame selector 380 performs the speckle tracking to find an area similar to the comparison area 5210 in a higher precision than the image pixel unit. The comparison frame selector 380 selects the matching area 5112 by performing the speckle tracking in the comparison area 5210 in the current frame and a similar area 5230 in the reference frame, which is obtained by the template matching. The right side of FIG. 5B shows an embodiment of performing the speckle tracking with the comparison area 5210 for the similar area 5230 selected from the reference frame in the comparison frame selector 380. An ultrasound RF signal for diagnosis that is irradiated by the ultrasound diagnosis device 20 includes a carrier frequency of an ultrasound wave. The characteristic of the carrier frequency may be used for a precise search at a precision equal to or greater than pixel unit resolution. The comparison frame selector 380 finds a similar area, i.e., a matching area 5260 (5112 of FIG. 5A), more correctly than a precision of the pixel unit resolution by using an ultrasound RF signal of the similar area 5250 and an ultrasound RF signal of the comparison area 5240.

The comparison frame selector 380 may calculate a movement displacement between the comparison area 5210 and the matching area 5260. In detail, the comparison frame selector 380 sets an arbitrary coordinate reference point in the current frame and calculates coordinates of the comparison area 5210. For example, the comparison frame selector 380 calculates coordinates C(Xc, Zc) of a central point of the comparison area 5210 by setting a depth from the skin of a patient (i.e., z-axis on the left side of FIG. 5B) and a lateral distance from a reference position of the ultrasound diagnosis device 20 (i.e., x-axis on the left side of FIG. 5B) as axes. Thereafter, the comparison frame selector 380 calculates coordinates R(Xc+Δx, Zc+Δz) of a central point of an area (5220) similar to the comparison area 5210 in the current frame that is selected by performing the template matching, wherein Δx and Δz denote pixel resolution. Thereafter, the comparison frame selector 380 calculates coordinates R′(Xc+Δx+δx, Zc+Δz+δz) of a central point of the matching area 5260 selected by performing the speckle tracking, with a precision equal to or greater than the pixel resolution. Accordingly, the comparison frame selector 380 derives a movement displacement (Δx+δx, Δz+δz) between the comparison area 5210 and the matching area 5260, with a precision equal to or greater than the pixel resolution.

An embodiment of calculating similarity between the comparison area 5111 and the matching area 5112 in operation 512 will now be described.

The similarity calculation (operation 512) expresses a similarity level between a current frame and each of the reference frames as a numerical value, and, for example, a similarity may be derived by calculating a correlation coefficient between the current frame and each reference frame. The correlation coefficient may be calculated using Pearson's formula as defined in Equation 1.

$$r = \frac{\text{?} \text{?} ( \text{?} - \overline{A} ) ( \text{?} - \overline{B} )}{\sqrt{ } ( \text{?} \text{?} {( \text{?} - \overline{A} )}^{2} ) ( \text{?} \text{?} {( \text{?} - \overline{B} )}^{2} )} \\ \text{?} \text{indicates text missing or illegible when filed} \qquad ( 1 )$$

In Equation 1, Aₘₙ denotes a value of a pixel at a horizontal mth position and a vertical nth position in the current frame. If the current frame and the reference frames are monochrome images, this pixel value may be a brightness value, and if the current frame and the reference frames are color images, this pixel value may be a color value. In detail, if it is assumed that a comparison area 531 selected in a current frame shown in FIG. 5C is divided into a predetermined number of pixels, Aₘₙ denotes a variable by which a value of a pixel 5311 at a horizontal mth position and a vertical nth position in the comparison area 531 is expressed by a predetermined corresponding value. In addition, Bₘₙ denotes a variable by which a value of an arbitrary pixel in a matching area 532 selected from a reference frame is expressed by a predetermined corresponding value, i.e., a variable indicating a pixel value of a pixel 5321 in the matching area 532 located at a position corresponding to that of the pixel 5311 located at the horizontal mth position and the vertical nth position in the comparison area 531. In addition, Ā denotes a mean value of pixel values of pixels forming a comparison area selected from the current frame. That is, if it is assumed that the comparison area 531 selected in the current frame shown in FIG. 5C is divided into a predetermined number of pixels, Ā denotes a mean value of pixel values of pixels forming the comparison area 531, which is defined as a representative value of the comparison area 531. In addition, B̅ denotes a mean value of pixel values of pixels forming a matching area selected from a reference frame, i.e., a mean value of the matching area 532 selected from the reference frame in a method corresponding to the method of obtaining Ā. A correlation coefficient r calculated by the comparison frame selector 380 using Equation 1 has a range of −1≦r≦1, and when the correlation coefficient r is 1 or −1, it is called a perfect correlation. In the current embodiment, selecting a reference frame having the most similarity to the current frame from among the reference frames as a comparison frame (operation 513) may indicate selecting a reference frame having a correlation coefficient r equal to or greater than 0.9 as a comparison frame.

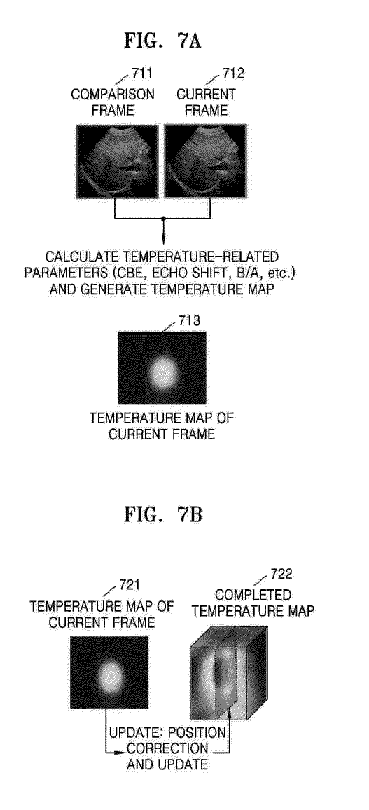

Operations of the comparator 350 and the temperature map generator 360 will now be described with reference to FIGS. 7A and 7B.

As described above, the comparator 350 generates a temperature map 713 of a current frame 712 by comparing echo signals forming the current frame 712 generated by the current frame generator 320 with echo signals forming a comparison frame 711 selected by the comparison frame selector 380 so that the temperature map generator 360 generates a completed temperature map for observing a temperature change in an observed part according to various criteria.

The temperature map generator 360 generates a completed temperature map 722 by using a temperature map 721 of a current frame, which is generated by the comparator 350. The temperature map 721 of the current frame displays a relative temperature change between observed parts of a comparison frame and the current frame in an image form, e.g., an image represented by different colors as reference numeral 721 of FIG. 7B or an image represented by different brightness values. The temperature map 721 of the current frame may be represented by a two-dimensional (2D) image or a 3D image.

After the temperature map 721 of the current frame is generated, the temperature map generator 360 generates the completed temperature map 722 for observing a temperature change in an observed part according to various criteria, as shown in FIG. 7B. In detail, the temperature map generator 360 generates the completed temperature map 722 by performing position correction and temperature map update using the generated temperature map 721 of the current frame. As an example of generating a completed temperature map using a temperature map of a current frame in the temperature map generator 360, the temperature map generator 360 may generate the completed temperature map 722 in which the entire observed area is represented as a 3D image by combining the 2D temperature map 721 of the current frame for a portion of the observed part with temperature maps generated for the remaining observed area in the same manner. In detail, the ultrasound diagnosis device 20 irradiates ultrasound waves while changing a position and orientation thereof under control of the driving device 60, and receives reflected waves of the irradiated ultrasound waves. Thereafter, the transducer 370 transduces the reflected waves into echo signals, the current frame generator 320 generates current frames that are a plurality of cross-sectional images of an observed part by using the echo signals, and the comparator 350 generates temperature maps of the current frames by comparing the generated current frames with reference frames. Thereafter, the temperature map generator 360 generates a completed temperature map with a 3D volume for three-dimensionally showing the observed part by accumulating these cross-sectional images. As such, a method of generating image data with a 3D volume by accumulating cross-sectional images is called a Multi-Planar Reconstruction (MPR) method.

As an example of generating a completed temperature map using a temperature map of a current frame in the temperature map generator 360, the temperature map generator 360 may sequentially accumulate the 2D temperature map 721 of the current frame for a portion of an observed part and 2D temperature maps of current frames for the same portion of the observed part according to an elapse of time. Accordingly, the temperature map generator 360 may generate a 2D completed temperature map in which an image change in a portion of an observed part according to an elapse of time is expressed. However, a completed temperature map generated by the temperature map generator 360 is not limited to the 3D completed temperature map for the entire observed part or the 2D completed temperature map in which an image change in a portion of an observed part according to an elapse of time is expressed by 2D temperature maps of current frames as described above, and may be generated as a 3D completed temperature map in which an image change in the entire observed part according to an elapse of time is expressed by accumulating 3D temperature maps of current frames for the entire observed part.

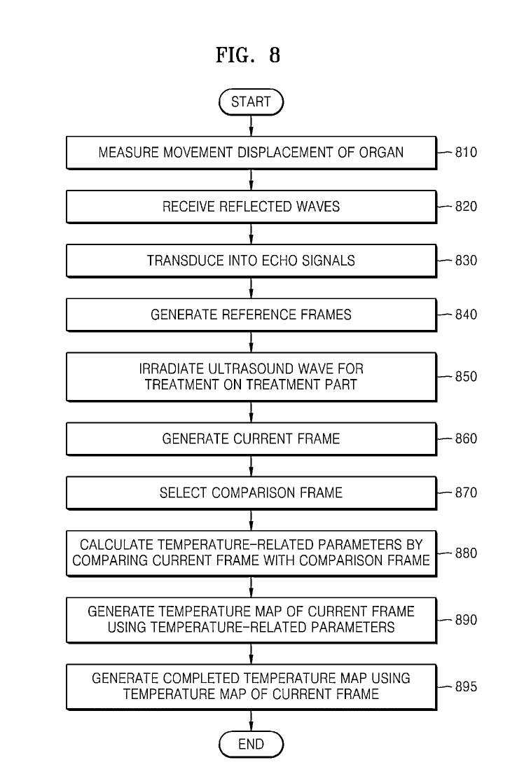

FIG. 8 is a flowchart illustrating a method of generating a temperature map of an organ using an ultrasound wave, according to an embodiment of the present disclosure.

Referring to FIG. 8, in operation 810, the controller 310 measures a movement displacement of a predetermined moving internal organ. In detail, the controller 310 measures a movement displacement of a predetermined internal organ of a patient moving in response to a breathing cycle of the patient.

In operation 820, the ultrasound diagnosis device 20 irradiates an ultrasound wave for diagnosis on an observed part in the predetermined moving internal organ by considering the measured movement displacement and receives reflected waves thereof. The ultrasound diagnosis device 20 irradiates the ultrasound wave for diagnosis in a range corresponding to the predetermined internal organ by considering the movement of the predetermined internal organ so that the observed part includes the entire treatment part 50.

In operation 830, the transducer 370 transduces the reflected waves received by the ultrasound diagnosis device 20 into echo signals.

In operation 840, the reference frame generator 330 generates reference frames indicating an image of the observed part by using the echo signals obtained from the transducer 370. In detail, the reference frame generator 330 generates reference frames indicating an image of the observed part by using the echo signals received from the transducer 370. Alternatively, a current frame generated at a time the ultrasound treatment device 10 irradiates an ultrasound wave for treatment on the treatment part 50 may be used as a reference frame. This may be implemented by updating the reference frame by the current frame in the reference frame generator 330, and a method of updating a reference frame by a current frame is as described above. In addition, although not shown in FIG. 8, the reference frame generator 330 may build a reference frame DB consisting of reference frames according to an embodiment of the present disclosure, as described above.

In operation 850, the ultrasound treatment device 10 irradiates the ultrasound wave for treatment on the treatment part 50 in the predetermined moving internal organ by considering the measured movement displacement.

In operation 860, the current frame generator 320 generates a current frame indicating a changed image of the observed part. In detail, the ultrasound diagnosis device 20 irradiates the ultrasound wave for diagnosis on the treatment part 50 and receives reflected waves thereof at the time the ultrasound treatment device 10 irradiates the ultrasound wave for treatment on the treatment part 50. The ultrasound diagnosis device 20 transmits the reflected waves to the transducer 370, and the transducer 370 transduces the reflected waves into echo signals and transmits the echo signals to the current frame generator 320. The current frame generator 320 generates a current frame indicating an image of the observed part by using the echo signals received from the transducer 370.

In operation 870, the comparison frame selector 380 selects a comparison frame that is a frame most similar to the current frame from among the reference frames. In addition, although not shown in FIG. 8, in a process of selecting the comparison frame, candidate reference frames may be selected from among reference frames in a reference frame DB by calculating an error in an estimated position and a breathing cycle, and a frame that is most similar to the current frame may be selected as the comparison frame from among the candidate reference frames, as described above.

In operation 880, the comparator 350 calculates temperature-related parameters indicating a relative temperature change between the current frame and the comparison frame by comparing echo signals forming the current frame with echo signals forming the comparison frame. The temperature-related parameters may be obtained in the CBE method, the ES method, or the B/A method, etc., as described above.

In operation 890, the comparator 350 generates a temperature map of the current frame by using the calculated temperature-related parameters. The temperature map of the current frame indicates a relative temperature change between the current frame and the comparison frame, as described above.

In operation 895, the temperature map generator 360 generates a completed temperature map indicating a temperature change in the observed part of the predetermined internal organ by using the temperature map of the current frame. The completed temperature map may be a 2D image or a 3D image at a predetermined time, or a 2D image or a 3D image that is changed over time, as described above.

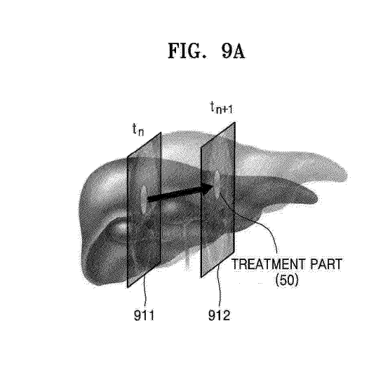

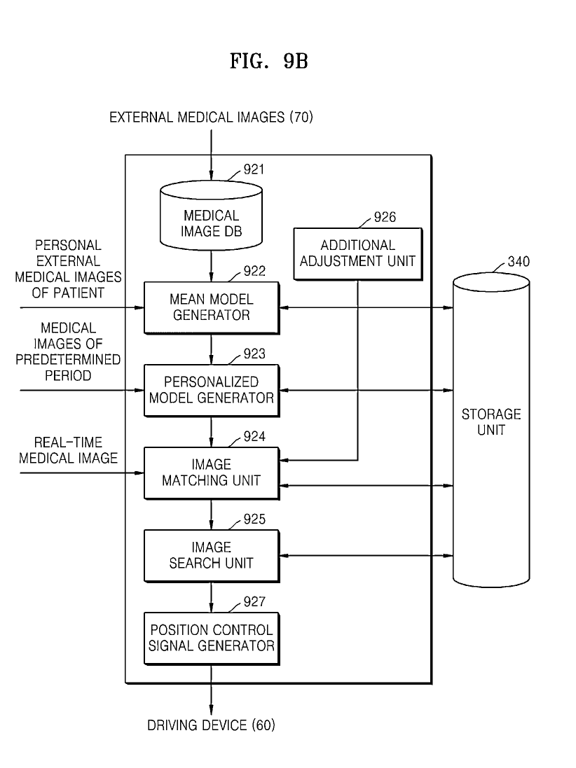

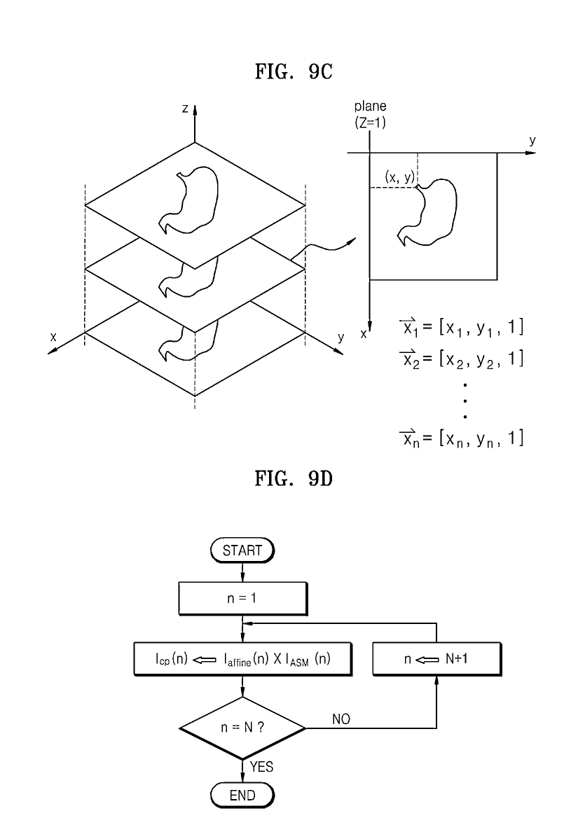

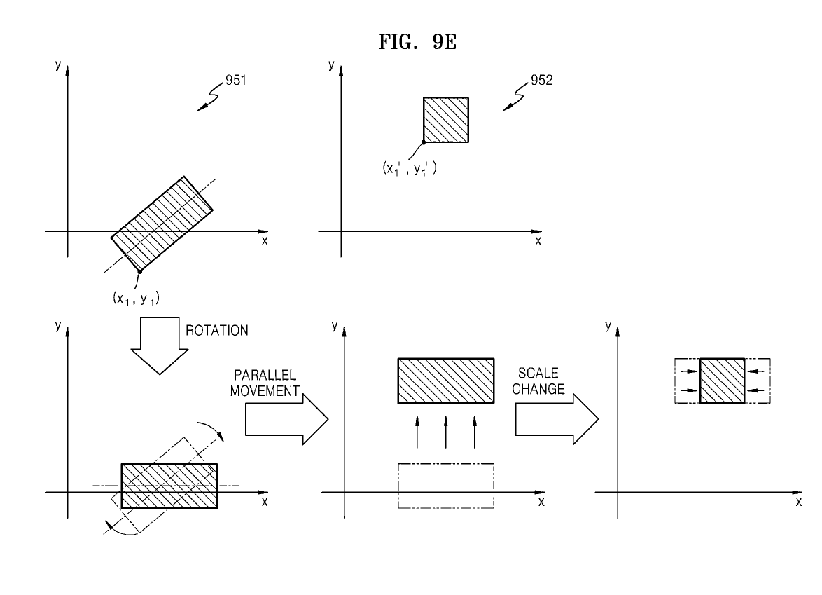

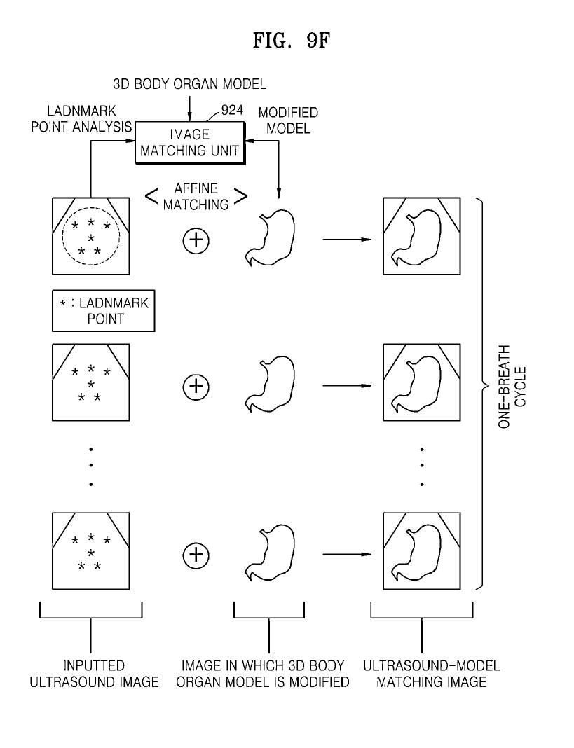

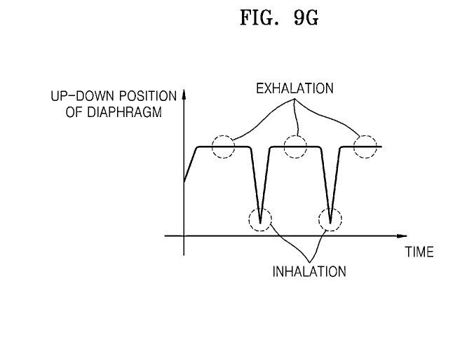

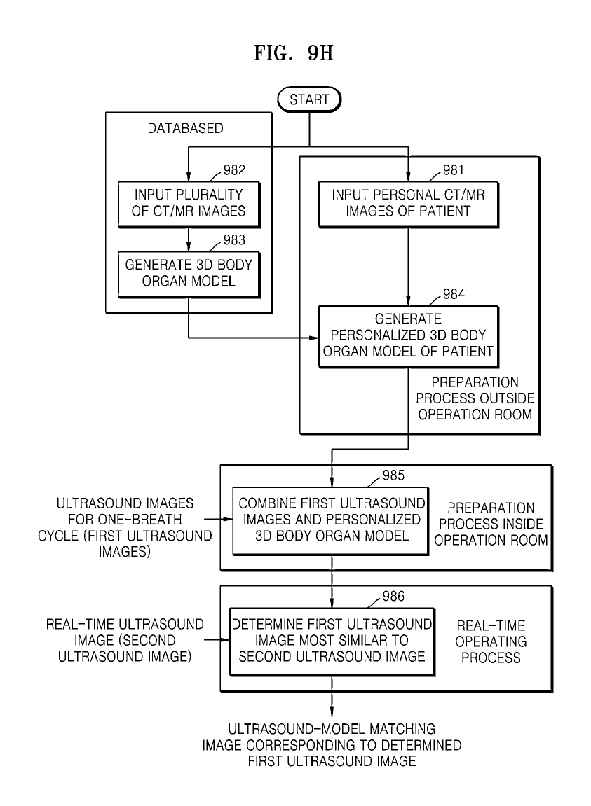

A method of measuring a temperature of a moving organ by using an ultrasound wave in an ultrasound treatment and diagnosis system for treating the moving organ in response to the movement of the internal organ of a human body, according to an embodiment of the present disclosure, will now be described with reference to FIGS. 9A to 9H, 10, and 11.

The current embodiment is characterized in that the ultrasound treatment device 10 irradiates the ultrasound wave for treatment on the treatment part 50 while tracking a displacement trajectory of the treatment part 50 that changes in correspondence with the movement displacement of an internal organ, and the ultrasound diagnosis device 20 irradiates the ultrasound wave for diagnosis on an observed part while tracking a displacement trajectory of the observed part and receives reflected waves thereof. To do as so, the controller 310 transmits position control signals for the ultrasound treatment device 10 and the ultrasound diagnosis device 20 to the driving device 60 according to the feature of the current embodiment. That is, if it is assumed that a predetermined time is tₙ as shown in FIG. 9A, the controller 310 correctly perceives a displacement trajectory of the treatment part 50 that changes in correspondence with the movement displacement of an internal organ at a next time tₙ₊₁, i.e., a displacement trajectory from reference numeral 911 to reference numeral 912. Thus, an embodiment of a method of generating an image suitable for rapid and accurate tracking of a predetermined internal organ including the treatment part 50 in medical images of a patient for a predetermined period will now be described.

FIG. 9B is a block diagram of the controller 310 shown in FIG. 2, according to an embodiment of the present disclosure. Referring to FIG. 9B, the controller 310 shown in FIG. 9B includes a medical image DB 921, a mean model generator 922, a personalized model generator 923, an image matching unit 924, an image search unit 925, an additional adjustment unit 926, and a position control signal generator 927.

The mean model generator 922 outputs a mean model of an organ to be treated by receiving and processing various personal medical images. In the current embodiment, the movement of an organ is tracked by generating a patient-personalized model, wherein generating a mean model is preparing to generate a personalized model, because the features of each patient need to be reflected to provide a correct operation environment to the patient because a shape, size, features, etc. of an organ vary according to an individual. To obtain a correct mean model, image information of various individuals may be used. In addition, even for an image obtained from each individual, images in various breathing motions may be obtained to reflect a shape of an organ changed in response to a breathing motion.

In detail, the mean model generator 922 receives images (hereinafter, external medical images 70) captured by medical experts for diagnosis of patients to analyze shapes, sizes, etc. of organs of various individuals, directly from a capturing device or from a storage medium storing the images. Thus, images that are easy to analyze contours of an organ and a lesion or the internal feature of an organ may be received. For example, Computed Tomography (CT) or Magnetic Resonance (MR) images may be received.

As a method of receiving external images, the external medical images 70 may be stored in a database by the medical image DB 921, and stored images may be retrieved. In the medical image DB 921, the external medical images 70 may be captured from various individuals by capturing devices and stored, or may be input from a storage medium. When images are retrieved from the medical image DB 921, all images may be retrieved, or some of the stored images may be retrieved according to a selection of a user.

As an embodiment, the mean model generator 922 may use a 3D Active Shape Models (ASM) algorithm based on the received external medical images 70. To use the ASM algorithm, the mean model generator 922 extracts shapes, sizes, and anatomic features of organs from the external medical images 70 by analyzing the external medical images 70 and generates a model obtained by statistically averaging the extracted shapes, sizes, and anatomic features of organs. The ASM algorithm is described in detail in “The Use of Active Shape Models For Locating Structure in Medical Images” (written by T. F. Cootes, A. Hill, C. J. Taylor and J. Haslam) published in 1994. By applying the ASM algorithm, a mean organ shape may be obtained, and this mean organ shape may be changed when a variable is adjusted.

FIG. 9C is a diagram for describing a process of analyzing the external medical images 70, i.e. a method of extracting position coordinate information of an organ boundary and an internal structure in the received CT or MR images. When a CT or MR Image is received, the mean model generator 922 applies different methods to a 2D image and a 3D image to extract position coordinate information of an organ boundary and an internal structure. The internal structure, for example, a liver, may include positions of a hepatic artery, a hepatic vein, a hepatic portal vein, and a hepatic duct, and may further include boundaries thereof.

When 2D images are received, image data with a 3D volume that three-dimensionally indicates a part to be extracted by accumulating a plurality of cross-sectional images is obtained to generate a 3D model, and this process is shown on the left side of FIG. 9C as a method of obtaining an image with a 3D volume by accumulating various pieces of image information. Three-dimensional coordinate information may be obtained by extracting position coordinate information of an organ boundary and an internal structure from a plurality of cross-sectional images before accumulation and adding coordinate information of an axis in an accumulating direction to the extracted position coordinate information, and because an image shown on the right side of FIG. 9C is an image of which a value on a z-axis is 1, z of a boundary position coordinate value extracted from the image is always 1. Thus, when coordinate information is extracted from cross-sectional images of image data on the left side of FIG. 9C, because the extracted coordinate information is 2D coordinate information, the extracted coordinate information is expressed as data of x- and y-axes. However, position coordinate information of a boundary is extracted as coordinates of [x, y, 1] by adding coordinate information of the z-axis to the data of the x- and y-axes. Then, the coordinate information becomes information including coordinates of the x-, y-, and z-axes. When a 3D image is received, position coordinate information of an organ boundary and an internal structure may be obtained by extracting cross-sectional images of the 3D image in a predetermined interval and performing the same process as a case of receiving 2D images. Extraction of boundary position coordinates from a 2D image in this process may be automatically or semi-automatically performed by an algorithm, or coordinate information may be manually input by a user based on displayed image information. For example, in a method of automatically obtaining boundary coordinate information, coordinate information of a point at which brightness in an image is rapidly changed may be obtained, and a position at which a frequency value is largest may be extracted as a boundary by using a Discrete Time Fourier Transform (DTFT). In a semi-automatic method, when information about some boundary points in an image is input by the user, neighboring boundary points may be extracted in the same method as the method of automatically obtaining coordinates based on the input boundary points. Because an organ boundary has a continuous and closed-curve shape, information about the entire boundary may be obtained using this nature. As such, because the entire image does not have to be searched in the semi-automatic method, a result may be more quickly obtained than in the automatic method. In a manual method, the user may directly designate coordinates of a boundary while viewing an image, and in this case, because designated intervals are not continuous, a boundary may be continuously extracted by interpolating discontinuous intervals in the middle. When position coordinate information of an organ and a lesion that is obtained in the disclosed methods is output by setting a brightness value of a voxel corresponding to the coordinates in a 3D space to a predetermined value, the user may view a shape of the organ and the internal structure expressed in a 3D graph. For example, if a brightness value of boundary coordinates of an organ to be checked is set to the minimum value, i.e., the darkest value, an image of the organ to be checked in an output image may be output dark, and if a brightness value of the organ to be checked is set to an intermediate value between a white color and a black color, and a brightness value of the coordinates of the lesion is set to the black color, the organ to be checked and the lesion may be easily discriminated from each other by the naked eye. Position coordinate information of a plurality of organ boundaries and internal structures obtained in this method may be defined as a data set and used as information for using the 3D ASM algorithm. The ASM algorithm will now be described.

To apply the ASM algorithm, coordinate axes of position coordinate information of a plurality of organ boundaries and internal structures are arranged to be in accord with each other. The arrangement of coordinate axes to be in accord with each other indicates that the centers of gravity of a plurality of objects to be arranged are moved to a single origin, and orientations of all organs in various shapes are rearranged. Thereafter, points used as landmark points are determined from the position coordinate information of the plurality of organ boundaries and internal structures. The landmark points are basic points for applying an algorithm. The landmark points are determined in the following methods:

1. A point at which the feature of an object is clearly reflected is determined as a landmark point. For example, in a case of a liver, points at which a blood vessel diverges, which commonly exist in all people, may be determined as landmark points, or in a case of a heart, a boundary at which the right atrium and the left atrium are divided and a boundary at which the main vein and the outer wall of the heart meet each other may be determined as landmark points.

2. The highest point or the lowest point of an object in a determined coordinate system is determined as a landmark point.

3. Points at which interpolation is performed between the points defined in 1. and 2. are determined as landmark points along a boundary in a predetermined constant interval.

When determined landmark points are in a 2D space, the landmark points may be expressed by x- and y-axes coordinates, and when determined landmark points are in a 3D space, the landmark points may be expressed by x-, y-, and z-axes coordinates. Thus, when determined landmark points are in a 3D space, if landmark point coordinates are expressed by vectors such as [symbol] x [symbol] , [symbol] x [symbol] , . . . , [symbol] x [symbol] (n denotes the number of landmark points), the vectors may be represented by Equation 2.

$${x}_{i 0} = [ {x}_{i 0} , {y}_{i 0} , {z}_{i 0} ] \\ {x}_{i 1} = [ {x}_{i 1} , {y}_{i 1} , {z}_{i 1} ] \\ ⋮ \\ {x}_{i n - 1} = [ {x}_{i n - 1} , {y}_{i n - 1} , {z}_{i n - 1} ] \qquad ( 2 )$$

The subscript i denotes position coordinate information of an organ boundary and an internal structure, which is obtained from an ith image. The number of pieces of position coordinate information may be large in cases, and in this case, the position coordinate information may be represented by a single vector to make computation of the position coordinate information easy. Then, a landmark point vector in which a total of the landmark points is represented by a single vector may be defined by Equation 3.

x [symbol] ᵢ=[xᵢ₀,yᵢ₀,zᵢ₀,xᵢ₁,yᵢ₁,zᵢ₁, . . . , x [symbol] ₘ₋₁,y [symbol] ₘ₋₁,z [symbol] ₘ₋₁]^{T} (3)

A size of the vector xᵢ is 3n× [symbol] 1.

When the number of data sets is N, a mean of landmark points in the total data sets may be represented by Equation 4.

$$\overline{x} = \frac{1}{N} {\sum }_{i = 1}^{N} {x}_{i} \qquad ( 4 )$$

Likewise, a size of the vector x̅ is 3n× [symbol] 1.

The mean model generator 922 obtains the mean landmark point x̅ by calculating Equation 4, and when a model is generated based on the mean landmark point x̅, the generated model may be a mean organ model. The ASM algorithm may not only generate a mean model but also change a shape of the mean model by adjusting a plurality of parameters. Thus, the mean model generator 922 not only simply calculates a mean model, but also calculates equations to apply a plurality of parameters.

The equations to apply a plurality of parameters will now be described.

A difference between a mean landmark point and each data may be represented by Equation 5. In Equation 5, the subscript i denotes an ith image. Thus, in Equation 5, a difference between a landmark point in each image and a mean landmark point of all images is obtained.

dxᵢ=xᵢ− x̅ (5)

A covariance matrix of x, y, and z may be defined by Equation 6 by using each data difference. Obtaining the covariance matrix is to obtain a unit eigenvector for the plurality of parameters for applying the ASM algorithm (detailed contents thereof is disclosed in the above-described paper).

$$S = \frac{1}{N} {\sum }_{i = 1}^{N} {dx}_{i} {dx}_{i}^{T} ( size is 3 n \times 3 n ) \qquad ( 6 )$$

If a unit eigenvector of the covariance matrix S is pₖ, the vector pₖ denotes an aspect in which a model generated by the ASM algorithm is modified. For example, a horizontal length of the model may be modified when a parameter b₁ multiplied by a vector p₁ is modified in a range of −2\sqrt{λ₁}≦b₁<2\sqrt{λ₁}, or a vertical length of the model may be modified when a parameter b₂ multiplied by a vector p₂ is modified in a range of −2\sqrt{λ₂}≦b₂<2\sqrt{λ₂}. The unit eigenvector pₖ (size is 3n×1) may be obtained by Equation 7.

Sₚ_{ₖ}=λₖpₖ (λₖ denotes an eigenvalue) (7)

Finally, a landmark point vector x to which modification is applied is calculated by using a mean landmark point vector x̅ as defined in Equation 8.

x= x̅+Pb (8)

In Equation 8, p=(p₁, p₂, . . . , pₜ) (size of each pₖ is 3n×1, and size of p is 3n×t) denotes first t eigenvectors, and b=(b₁, b₂, . . . , bₜ)^{T} (size is t×1) denotes a weight of each eigenvector.

The mean model generator 922 may calculate x̅ (size is 3n×1) indicating a mean model shape and the vector p=(p₁, p₂, . . . , pₜ) (size is 3n×t) for applying modification using the 3D ASM algorithm through the equations described above.

The personalized model generator 923 receives the mean organ model x̅ and the vector p=(p₁, p₂, . . . , pₜ) (size is 3n×t) from the mean model generator 922 and generates a personalized model by processing parameters in the 3D ASM algorithm. Because organs of individual patients are also different in shapes and sizes, if the mean organ model is used as it is, accuracy may decrease because individuals have their own features such that an organ is horizontally longer, vertically longer, thicker on the left, or lower on the right than a mean shape. In addition, when there is a lesion in an organ of an individual, the personalized model generator 923 may insert a position of the lesion into a model to correctly perceive a shape and position of the lesion. Thus, the personalized model generator 923 receives the external medical images of an individual patient from an image capturing device or a storage medium, analyzes personal organ shape, size, and position information, and if there is a lesion, analyzes position, size, and shape information of the lesion. This process will now be described in detail.

The personalized model generator 923 determines a weight (vector b) of an eigenvector in the ASM algorithm for an individual patient based on an image on which a shape of an organ, such as a CT or MR image, is clearly perceived. Thus, first, the external medical images 70 of the individual patient are received, and position coordinate information of an organ boundary and an internal structure is perceived using the process of FIG. 9C as in the process of analyzing the external medical images 70 in the mean model generator 922. Furthermore, if landmark point coordinate information is perceived in the same process as the method of perceiving landmark points when the algorithm is applied, a value of a vector x (size is 3n×1) that is a patient-personalized landmark point set may be obtained. An organ model generated based on the vector x may be a personalized model. Equation 9 may be obtained by applying the nature of an inverse function and a unit eigenvector (pₖ^{T}pₖ=1) to Equation 8. A value of b=(b₁, b₂, . . . , bₜ)^{T} is determined by Equation 9.

b=P^{T}(x− x̅) (9)

The information about the vectors x̅ and p determined by the mean model generator 922 may be stored in the storage unit 340 as a mean organ model in a DB to be repeatedly used. In addition, the external medical images 70 of an individual patient that are input in the personalized model generator 923 may undergo a learning process added when a mean model stored in the DB is determined for a medical examination of a next patient.

The image matching unit 924 receives information about the vectors x, x̅, p, and b from the personalized model generator 923 and matches the received vector information with medical images of a patient for a predetermined period. The matching indicates that a model using the ASM algorithm overlaps with an ultrasound image at a position of an organ in the ultrasound image and is output, and more correctly, a pixel or voxel value corresponding to coordinate information of a model formed by the ASM algorithm may be replaced by a predetermined brightness value or may overlap with the coordinate information. When the replacement is performed, only a personalized model may be output by removing an organ part from an original ultrasound image. However, when the overlapping is performed, an image in which the original ultrasound image and the personalized model overlap each other may be output. The overlapped image is easy to identify with the naked eye if different colors are used in the overlapped image. For example, when a blue personalized model overlaps with a monochrome ultrasound image, a graphic figure may be easily identified by the naked eye.

The medical image may be a real-time captured image, e.g., an ultrasound image. The medical image may be a 2D or 3D image. The predetermined period may be a one-breath cycle because a change in an organ may have a constant period during a breathing cycle of a human body. For example, when a one-breath cycle of a patient is 5 seconds, if an ultrasound image of 20 frames per second (fps) is generated, an image of a total of 100 frames may be generated.

A process of matching an image in the image matching unit 924 may be largely divided into two operations: reflecting a change in an organ due to breathing in an ultrasound image input for a predetermined period in a 3D organ model; and aligning the modification-reflected 3D organ model with a corresponding organ in the ultrasound image by performing scale adjustment, axis rotation, and axis movement of the modification-reflected 3D organ model.

The operation of reflecting a change in an organ due to breathing in a 3D organ model will now be described. For example, in a case of an ultrasound image before matching with a medical image, a value of a vector b that is a weight value, a parameter of the ASM algorithm, is adjusted by perceiving a position and change of the organ according to frames of the ultrasound image. The adjusted value of the vector b is not much different from the value of the vector b determined by the mean model generator 922. The reason is because the image matching unit 924 reflects only the change due to breathing of a patient, wherein a shape change in an organ due to breathing is less than a difference from another individual, i.e., another person. Thus, when the value of the vector b is determined by the image matching unit 924, only a change within a predetermined limited range is added based on the value of the vector b determined by the mean model generator 922. In addition, a vector b of a previous frame may be reflected to determine a vector b of a next frame because a large change does not occur for a short period between frames because a change in an organ in a breathing process is continuous. After the value of the vector b is determined, a personalized model in which a change in the organ is reflected in each ultrasound image may be generated according to frames by computation of the 3D ASM algorithm.

FIG. 9D is a flowchart illustrating a process of matching a personalized model in which a change in an organ is reflected in each image with a position of the organ in an ultrasound image through rotation, scale adjustment, and parallel movement in the image matching unit 924, according to an embodiment of the present disclosure. In detail, FIG. 9D is a flowchart in which when the vector b that is a weight value of an eigenvector is determined for each frame, a one-to-one affine registration is performed for each frame. If it is assumed that the number of frames is N, and a frame number is n, one-to-one matching is performed from n=1 until n=N. An affine transform function T_{affine} is acquired using an Iterative Closest Point (ICP) algorithm for each frame based on a landmark point set in the ultrasound image and a landmark point set in the personalized model, and a 3D human body organ model image is transformed using the acquired affine transform function T_{affine}. The ICP algorithm is an algorithm of performing rotation, parallel movement, and scale adjustment of the remaining images based on one image to align the same objects in a plurality of images. The ICP algorithm is described in detail in “Iterative point matching for registration of free-form curves and surfaces” (written by Zhengyou Zhang).

FIG. 9E schematically illustrates a method of acquiring the affine transform function T_{affine} from a 2D image. Reference numeral 951 denotes a state before an affine transform is applied, and reference numeral 952 denotes a state after the affine transform is applied. Although rotation, parallel movement, and scale adjustment are performed when the affine transform is applied, if first coordinates and final coordinates are acquired by Equation 10 using the fact that the affine transform is one-to-one point correspondence, a coefficient of a matrix T_{affine} may be directly determined.

$$[ \begin{matrix}{x}_{1}^{′} \\ {y}_{1}^{′}\end{matrix} ] = {T}_{affine} [ \begin{matrix}{x}_{1} \\ {y}_{1} \\ 1\end{matrix} ] = [ \begin{matrix}{a}_{1} & {b}_{1} & {c}_{1} \\ {a}_{2} & {b}_{2} & {c}_{2}\end{matrix} ] [ \begin{matrix}{x}_{1} \\ {y}_{1} \\ 1\end{matrix} ] \qquad ( 10 )$$

Equation 11 is an equation for applying an affine transform function T_{affine} acquired from a 3D space or above instead of a 2D space to each frame.

x_{ICP}(n)=T_{affine}(n)×x_{ASM}(n) (11)

In Equation 11, n denotes an nth frame and is an integer (1≦n≦N). In addition, x_{ASM}(n) denotes a landmark point vector obtained by changing the vector b that is a weight value in the image matching unit 924. According to the formed x_{ICP}(n), when position coordinate information of an organ boundary and an internal structure on which a change is reflected for each frame is matched with an ultrasound image, if a voxel value corresponding to the position coordinate information in the ultrasound image may be replaced by a predetermined brightness value or may overlap with the position coordinate information, a graphic figure of an organ may be identified by the naked eye.

FIG. 9F is a diagram for describing an image matching process in the image matching unit 924. FIG. 9F shows a process of forming matching images between medical images input for a predetermined period and a human body organ model in the image matching unit 924 based on ultrasound images input for a one-breath cycle. The input ultrasound images are arranged on the left side of FIG. 9F, wherein * denotes a landmark point in the input ultrasound images. The input ultrasound images may reflect various patterns of a breathing motion from inhalation to exhalation.

The personalized model generated by the personalized model generator 923 may be changed in a shape thereof according to a breathing motion. However, the change according to a breathing motion will be less than a change due to the variety between individuals. Thus, when the change according to a breathing motion is reflected, a method of adjusting a parameter value determined by the personalized model generator 923 may be quicker and easier than newly obtaining a parameter value in the 3D ASM algorithm. The affine transform function T_{affine} using the ICP algorithm is applied using landmark points in an organ model and landmark points in an organ of an ultrasound image on which the change is reflected. Through the affine transform, a size and position of a 3D organ model may be changed to meet a size and position of the organ in the ultrasound image. Synthesizing the changed model with the ultrasound image may be performed by a method of replacing a pixel (or voxel) value of the ultrasound image that corresponds to a position of the changed model by a predetermined value or overlapping the pixel (or voxel) value of the ultrasound image with the changed model. The matched image is called an ultrasound-model matching image and may be stored in the storage unit 340.

The image search unit 925 performs a process in a surgery. In brief, a graphic figure of an organ in a real-time input ultrasound image is displayed on a screen, and a surgeon performs the surgery while viewing the graphic figure with the naked eye. This process will now be described in detail. First, a real-time medical image of a patient is received. In this case, the medical image may be the same image as received from the image matching unit 924. Thus, if an ultrasound image is used as an example like the above example, when a real-time ultrasound image is received, the received ultrasound image is compared with medical images received from the image matching unit 924 for a predetermined period to determine the most similar image, and an ultrasound-model matching image corresponding to the determined image is searched for in the storage unit 340 and output.

An embodiment of comparing similar images from among ultrasound images is a method of determining an image by detecting a position of a diaphragm. If a position of a diaphragm in the received real-time medical image is X, a difference between a position of a diaphragm in each of a plurality of medical images received by the image matching unit 924 for a predetermined period and X, and an image having the least difference is detected. FIG. 9G is a graph showing the movement of a diaphragm of which an absolute position moves upwards and downwards. Analyzing this graph, the position regularly moves in response to a breathing cycle. When medical images received for a predetermined period by the image matching unit 924 and real-time medical images received by the image search unit 925 are captured, a position of the ultrasound diagnosis device 20 and a position of a patient may be fixed, because when the position of the ultrasound diagnosis device 20 or the position of the patient is changed, a relative position of an organ in an image may be changed, and in this case, an accurate and rapid search of an image cannot be performed in image comparison.

An embodiment of comparing similar images from among ultrasound images is a method of determining an image by using a pixel brightness difference. That is, this is a method using that a brightness difference between most similar images is the least. In detail, when an image (second image) of a single frame in a real-time medical image is searched for from among medical images (first images) for a predetermined period that are used for the matching, a brightness difference between any one of the first images and the second image is first calculated, and a variance based on a total brightness difference is obtained. Then, variances are obtained between the remaining first images and the second image in the same way, and the most similar image may be determined by determining an image having the least variance.

The additional adjustment unit 926 may adjust a final output result by adjusting the affine transform function T_{affine} and the parameters of the 3D ASM algorithm by the user while the user views a displayed image. That is, the user performs a correct transform with the naked eye while viewing a displayed image.

FIG. 9H is a flowchart illustrating a method of dynamically tracking an organ and a lesion based on a 3D organ model, according to an embodiment of the present disclosure. Operations 982 and 983 may be already-processed databases. In operation 982, CT or MR images for various breathing cycles of various individuals are received. In operation 983, a 3D human body organ model is generated based on the received images, wherein the 3D ASM algorithm may be used as described above.

In operation 981, CT or MR images of a patient are received. In operation 984, the 3D human body organ model generated in operation 983 is modified based on the images received in operation 981. The process of generating a personalized 3D human body organ model may be performed even outside an operation room. In operation 985, ultrasound images for a one-breath cycle of the patient (hereinafter, referred to as first ultrasound images) are received, and the first ultrasound images are matched with the personalized 3D human body organ model. The matched images are called ultrasound-model matching images, and may be stored in a temporary memory or a storage medium such as the storage unit 340. Operation 985 may be performed as a preparation process inside the operation room. In addition, positions of the patient and a probe in operations 985 and 986 may be fixed. In operation 986 as a real-time operation in the operation room, when a real-time ultrasound image of the patient (a second ultrasound image) is received, a first ultrasound image most similar to the second ultrasound image is determined, and an ultrasound-model matching image corresponding to the determined first ultrasound image, i.e., an image of a predetermined moving internal organ including the treatment part 50, is generated.

The position control signal generator 927 receives the ultrasound-model matching image generated by the image search unit 925, i.e., an image of a predetermined moving internal organ including the treatment part 50, from the image search unit 925 and generates position control signals for the ultrasound treatment device 10 and the ultrasound diagnosis device 20 in response to the received image. Thereafter, the position control signal generator 927 transmits the generated position control signals to the driving device 60. Accordingly, the ultrasound treatment device 10 may irradiate an ultrasound wave for treatment on the treatment part 50 along with the movement of the internal organ of the patient, and the ultrasound diagnosis device 20 may irradiate an ultrasound wave for diagnosis on the observed part along the movement of the internal organ of the patient and receive reflected waves thereof.

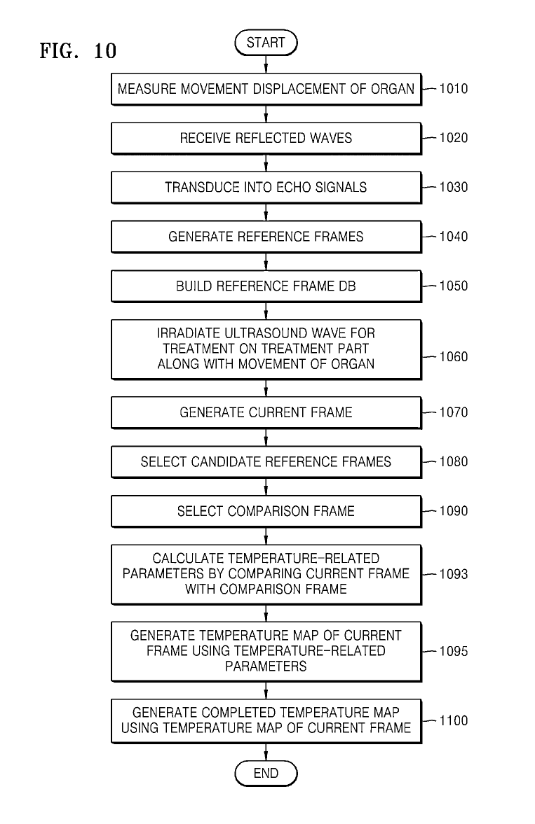

FIG. 10 is a flowchart illustrating a method of generating a temperature map of a moving organ using an ultrasound wave in an ultrasound treatment and diagnosis system for treating a patient in response to the movement of an internal organ, according to an embodiment of the present disclosure.

Referring to FIG. 10, in operation 1010, the controller 310 measures a movement displacement of a predetermined moving internal organ. In detail, the controller 310 measures a movement displacement of a predetermined internal organ of the patient moving in response to a breathing cycle of the patient.

In operation 1020, the ultrasound diagnosis device 20 irradiates an ultrasound wave for diagnosis on an observed part in the predetermined moving internal organ by considering the measured movement displacement and receives reflected waves thereof. The ultrasound diagnosis device 20 irradiates the ultrasound wave for diagnosis in a range corresponding to the predetermined internal organ by considering the movement of the predetermined internal organ so that the observed part includes the entire treatment part 50.

In operation 1030, the transducer 370 transduces the reflected waves received by the ultrasound diagnosis device 20 into echo signals.

In operation 1040, the reference frame generator 330 generates reference frames indicating an image of the observed part. In detail, the reference frame generator 330 generates reference frames indicating an image of the observed part by using the echo signals received from the transducer 370. In general, the reference frames are generated as frames including temperature information of the observed part before the ultrasound treatment device 10 irradiates the ultrasound wave for treatment on the treatment part 50. Alternatively, a current frame generated at a time the ultrasound treatment device 10 irradiates an ultrasound wave for treatment on the treatment part 50 may be used as a reference frame. This may be implemented by updating the reference frame by the current frame in the reference frame generator 330, and a method of updating a reference frame by a current frame is as described above.

In operation 1050, the reference frame generator 330 builds a reference frame DB with one or more reference frames

In operation 1060, the ultrasound treatment device 10 irradiates the ultrasound wave for treatment on the treatment part 50 along with the movement of the treatment part 50 in the predetermined internal organ in response to the position control signal transmitted from the controller 310 to the driving device 60 based on the ultrasound-model matching image generated by the controller 310, i.e., an image of a predetermined moving internal organ including the treatment part 50.

In operation 1070, the current frame generator 320 generates a current frame indicating a changed image of the observed part. In detail, the ultrasound diagnosis device 20 irradiates the ultrasound wave for diagnosis on the treatment part 50 and receives reflected waves thereof at the time the ultrasound treatment device 10 irradiates the ultrasound wave for treatment on the treatment part 50. For this operation, the ultrasound diagnosis device 20 also needs to move in response to the position control signal transmitted from the controller 310 to the driving device 60 based on the ultrasound-model matching image generated by the controller 310, i.e., an image of a predetermined moving internal organ including the treatment part 50. The ultrasound diagnosis device 20 transmits the reflected waves to the transducer 370, and the transducer 370 transduces the reflected waves into echo signals and transmits the echo signals to the current frame generator 320. The current frame generator 320 generates a current frame indicating an image of the observed part by using the echo signals received from the transducer 370.

In operation 1080, the reference frame generator 330 selects candidate reference frames from among the reference frames in the built reference frame DB by calculating errors in an estimated position and a breathing cycle.

In operation 1090, the comparison frame selector 380 selects a comparison frame that is a frame most similar to the current frame from among the candidate reference frames.

In operation 1093, the comparator 350 calculates temperature-related parameters indicating a relative temperature change between the current frame and the comparison frame by comparing the current frame with the comparison frame. The temperature-related parameters may be obtained in the CBE method, the ES method, or the B/A method, etc., as described above.

In operation 1095, the comparator 350 generates a temperature map of the current frame by using the calculated temperature-related parameters. The temperature map of the current frame indicates a relative temperature change between the current frame and the comparison frame, as described above.

In operation 1100, a completed temperature map indicating a temperature change in the observed part of the predetermined internal organ is generated by using the temperature map of the current frame. The completed temperature map may be a 2D image or a 3D image at a predetermined time, or a 2D image or a 3D image that is changed over time, as described above.

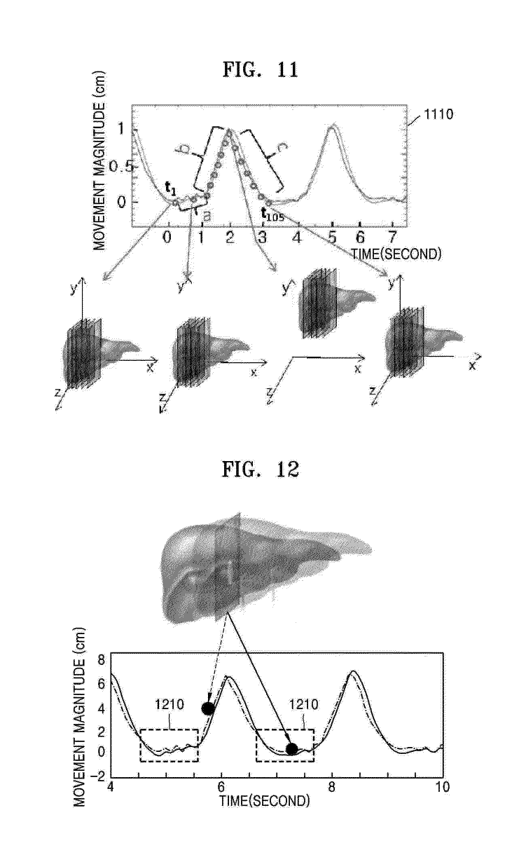

FIG. 11 is a diagram for describing constructing a reference frame DB by the reference frame generator 330 (operation 1050) in an HIFU system for treating an internal organ along with the movement of the internal organ, according to an embodiment of the present disclosure. In detail, an example of showing the movement displacement of the organ over time is shown as a graph 1110. When a time of summing periods a, b, and c is a one-breath cycle in the graph 1110, the periods a, b, and c indicate a pause period between a breathing motion, an inhalation period, and an exhalation period, respectively. When the reference frame generator 330 generates reference frames for a one-breath cycle, because the pause period between a breathing motion has a relatively smaller movement magnitude of the organ than the inhalation period and the exhalation period, the number of reference frames generated during the pause period by the reference frame generator 330 may be relatively less than those generated during the inhalation period or the exhalation period. An example will now be made to describe the building of the reference frame DB (operation 1050). As shown in the graph 1110, it is assumed that a one-breath cycle is t₁ to t₁₀₅. In addition, it is assumed that the period a is a pause period between a breathing motion, wherein a movement magnitude of the organ is measured as approximately 1 mm, and the periods b and c are inhalation and exhalation periods, respectively, wherein each movement magnitude of the organ is measured as approximately 5 mm. In addition, it is assumed that reference frames of 50 frames per point are needed to build a proper reference frame DB including the treatment part 50. Here, the point indicates a location at which a reference frame is acquired in correspondence with a movement magnitude of the organ in each period. If one point is needed every time the organ moves by 0.2 mm, a total of 5 points are needed during the period a (the pause period between a breathing motion), and 50 points are needed during each of the period b (the inhalation period) and the period c (the exhalation period). Thus, reference frame acquisition places of a total of 105 points are needed for a one-breath cycle. As a result, the number of reference frames stored in the reference frame DB for a one-breath cycle is 5250. This embodiment is only illustrative, and it will be understood by one of ordinary skill in the art that the number of reference frames may be calculated in another way only if the same principle is applied.

A method of generating a temperature map of an internal organ using an ultrasound wave in an ultrasound treatment and diagnosis system for treating the internal organ in a pause between a breathing motion, according to an embodiment of the present disclosure, will now be described with reference to FIGS. 12, 13, and 14.

An HIFU therapy for treating an internal organ in a pause between a breathing motion indicates that the therapy is performed only in the pause between a breathing motion in which the movement of the organ is minimized instead of a therapy performed in all periods of a breathing motion. In detail, a one-breath cycle consists of a pause period between a breathing motion, an inhalation period, and an exhalation period, wherein the movement displacement of the internal organ is relatively smaller in the pause period between a breathing motion than in the inhalation period or the exhalation period to be more effective to irradiate an ultrasound wave on a predetermined treatment part 50. Referring to FIG. 12, a period in which the movement displacement of the internal organ is relatively small in a breathing cycle is called a pause between a breathing motion (referred to as 1210), and the current embodiment is characterized in that the ultrasound treatment device 10 irradiates the ultrasound for treatment on the treatment part 50 in the pause between a breathing motion. The pause between a breathing motion is derived from the movement displacement of a predetermined moving internal organ that is measured by the controller 310. The ultrasound treatment and diagnosis system for treating an internal organ in a pause between a breathing motion according to the current embodiment may be implemented in both cases where the ultrasound treatment device 10 and the ultrasound diagnosis device 20 are physically movable and where the ultrasound treatment device 10 and the ultrasound diagnosis device 20 are physically fixed.

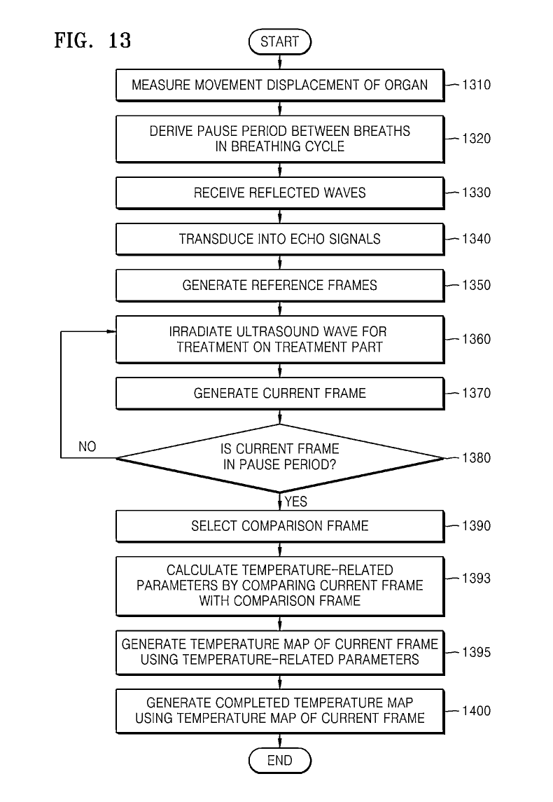

FIG. 13 is a flowchart illustrating a method of measuring a temperature of an internal organ using an ultrasound wave in an ultrasound treatment and diagnosis system for treating the internal organ in a pause between a breathing motion, according to an embodiment of the present disclosure.

Referring to FIG. 13, in operation 1310, the controller 310 measures a movement displacement of a predetermined moving internal organ. In detail, the controller 310 measures a movement displacement of a predetermined internal organ of a patient moving in response to a breathing cycle of the patient.

In operation 1320, the controller 310 derives a pause period between a breathing motion from the measured movement displacement of the predetermined internal organ of the patient In operation 1330, the ultrasound diagnosis device 20 irradiates an ultrasound wave for diagnosis on an observed part in the predetermined moving internal organ by considering the measured movement displacement and receives reflected waves thereof. The ultrasound diagnosis device 20 irradiates the ultrasound wave for diagnosis in a range corresponding to the predetermined internal organ by considering the movement of the predetermined internal organ so that the observed part includes the entire treatment part 50.

In operation 1340, the transducer 370 transduces the reflected waves received by the ultrasound diagnosis device 20 into echo signals.

In operation 1350, the reference frame generator 330 generates reference frames indicating an image of the observed part by using the echo signals. In detail, the reference frame generator 330 generates reference frames indicating an image of the observed part by using the echo signals received from the transducer 370. In general, the reference frames are generated as frames including temperature information of the observed part before the ultrasound treatment device 10 irradiates the ultrasound wave for treatment on the treatment part 50. Alternatively, a current frame generated at a time the ultrasound treatment device 10 irradiates an ultrasound wave for treatment on the treatment part 50 may be used as a reference frame. This may be implemented by updating the reference frame by the current frame in the reference frame generator 330, and a method of updating a reference frame by a current frame is as described above. In addition, although not shown in FIG. 13, the reference frame generator 330 may build a reference frame DB consisting of reference frames according to an embodiment of the present disclosure, as described above.

In operation 1360, the ultrasound treatment device 10 irradiates the ultrasound wave for treatment on the treatment part 50 in the predetermined moving internal organ during the derived pause period between a breathing motion.

In operation 1370, the current frame generator 320 generates a current frame indicating a changed image of the observed part. In detail, the ultrasound diagnosis device 20 irradiates the ultrasound wave for diagnosis on the treatment part 50 and receives reflected waves thereof at the time the ultrasound treatment device 10 irradiates the ultrasound wave for treatment on the treatment part 50. The ultrasound diagnosis device 20 transmits the reflected waves to the transducer 370, and the transducer 370 transduces the reflected waves into echo signals and transmits the echo signals to the current frame generator 320. The current frame generator 320 generates a current frame indicating an image of the observed part by using the echo signals received from the transducer 370. The current frame includes information about a position and temperature of the observed part. The information about the temperature may be expressed by displaying a temperature distribution on the observed part with different colors or different brightness values.

In operation 1380, the current frame generator 320 determines whether the generated current frame is a frame generated during the pause period between a breathing motion. If the generated current frame is a frame generated during the pause period between a breathing motion, the method proceeds to operation 1390. Otherwise, if the generated current frame is a frame generated except for the pause period between a breathing motion, the method proceeds back to operation 1360 to perform operations 1360 and 1370 again.

In operation 1390, the comparison frame selector 380 selects a comparison frame that is a frame most similar to the current frame from among the reference frames. In addition, although not shown in FIG. 13, in a process of selecting the comparison frame, candidate reference frames may be selected from among reference frames in the reference frame DB by calculating an error in an estimated position and a breathing cycle, and a frame that is most similar to the current frame may be selected as the comparison frame from among the candidate reference frames, as described above.

In operation 1393, the comparator 350 calculates temperature-related parameters indicating a relative temperature change between the current frame and the comparison frame by comparing the current frame with the comparison frame. The temperature-related parameters may be obtained in the CBE method, the ES method, or the B/A method, etc., as described above.

In operation 1395, the comparator 350 generates a temperature map of the current frame by using the calculated temperature-related parameters. The temperature map of the current frame indicates a relative temperature change between the current frame and the comparison frame, as described above.

In operation 1400, the temperature map generator 360 generates a completed temperature map indicating a temperature change in the observed part of the predetermined internal organ by using the temperature map of the current frame. The completed temperature map may be a 2D image or a 3D image at a predetermined time, or a 2D image or a 3D image that is changed over time, as described above.

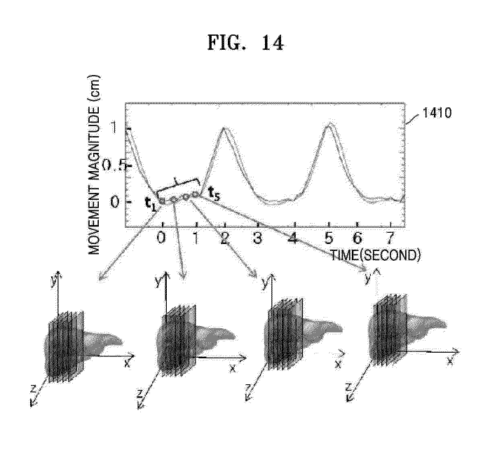

FIG. 14 is a diagram for describing constructing a reference frame DB in the reference frame generator 330 in the ultrasound treatment and diagnosis system for treating an internal organ in a pause between a breathing motion (operation 1350), according to an embodiment of the present disclosure. Although not shown in FIG. 13, the reference frame DB may be built by the reference frames generated by the reference frame generator 330, as described above. In detail, an example of showing the movement displacement of the organ over time as a graph 1410. In the graph 1410, a period between t₁ and t₅ indicates a pause period between a breathing motion. The building of the reference frame DB will now be described as an example. As shown in the graph 1410, it is assumed that the period between t₁ and t₅ is a pause period between a breathing motion, wherein a movement magnitude of the organ is measured as approximately 1 mm. In addition, it is assumed that reference frames of 50 frames per point are needed to build a proper reference frame DB including the treatment part 50 by the reference frame generator 330. Here, the point indicates a place at which a reference frame is acquired in correspondence with a movement magnitude of the organ in each period, as described above. If one point is needed every time the organ moves by 0.2 mm, a total of 5 points are needed during the period between t₁ and t₅ (a pause between a breathing motion). Thus, reference frame acquisition locations of a total of 5 points are needed, and the number of reference frames stored in the reference frame DB is 250. This embodiment is only illustrative, and it will be understood by one of ordinary skill in the art that the number of reference frames may be calculated in another way only if the same principle is applied.

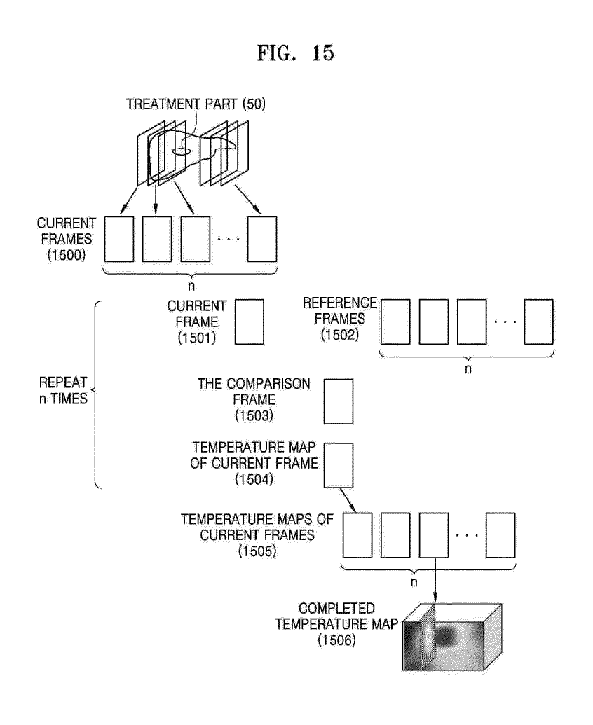

A method of generating a temperature map that is characterized in that the ultrasound diagnosis device 20 operates at a fixed position thereof in an ultrasound treatment and diagnosis system for treating an internal organ, according to an embodiment of the present disclosure, will now be described with reference to FIG. 15

The current embodiment corresponds to a method of irradiating the ultrasound wave for diagnosis on an observed part in a physically fixed state. Thus, because the observed part with respect to which the ultrasound diagnosis device 20 irradiates the ultrasound wave for diagnosis and receives reflected waves thereof at a time the ultrasound treatment device 10 irradiates the ultrasound wave for treatment may not include the treatment part 50, a current frame generated by the current frame generator 320 may also not include an image of the treatment part 50. Therefore, in the current embodiment, a process of generating a plurality of current frames 1500 for the entire predetermined internal organ including the treatment part 50 is needed. A detailed description according to the current embodiment describes a process of generating a temperature map 1504 of a current frame 1501 that is one of the plurality of current frames 1500. In the current embodiment in which the plurality of current frames 1500 are generated, the temperature map 1504 of a current frame that corresponds to each current frame by repeating a process described below.

First, reference frames 1502 of a predetermined internal organ including the treatment part 50 are generated for a one-breath cycle of a patient. A detailed method of generating the reference frames 1502 is as described above.

Thereafter, the current frame generator 320 generates the current frame 1501 at a time the ultrasound treatment device 10 irradiates the ultrasound wave for treatment. A detailed method of generating the current frame 1501 is as described above. Thereafter, the comparison frame selector 380 selects a comparison frame 1503 corresponding to the current frame 1501 from among the reference frames 1502. Thereafter, the comparator 350 generates the temperature map 1504 of the current frame 1501 by using the current frame 1501 and the comparison frame 1503 corresponding to the current frame 1501.

Thereafter, the temperature map generator 360 generates a completed temperature map 1506 with a 3D volume that three-dimensionally shows the predetermined internal organ including the treatment part 50 by accumulating temperature maps 1505 of current frames generated for each current frame by repeating the above process. A method of operating the current frame generator 320, the comparison frame selector 380, the comparator 350, and the temperature map generator 360 is as described above.

As described above, according to the one or more of the above embodiments of the present disclosure, even when an internal organ is moving, a temperature change at a predetermined part of the organ according to ultrasound irradiation may be correctly measured. In addition, in a case of an ultrasound therapy for treating a disease in a method of irradiating an ultrasound wave along with a moving organ, a necrosis level of tissue in a treatment part may be correctly perceived by correctly measuring a temperature change at the treatment part. In addition, even in a case of an ultrasound therapy for treating a disease in a method of irradiating an ultrasound wave during a pause between a breathing motion, a necrosis level of tissue in a treatment part may be correctly perceived by correctly measuring a temperature change at the treatment part. In particular, because a temperature of a treatment part is correctly monitored even in a high temperature range while an ultrasound therapy is performed, the ultrasound therapy may be efficiently performed such that a treating time is shortened. In addition, a treatment part and normal surrounding tissue may be prevented from being damaged by an ultrasound wave for treatment.

The above-described embodiments may be recorded in computer-readable media including program instructions to implement various operations embodied by a computer. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. The computer-readable media may also be a distributed network, so that the program instructions are stored and executed in a distributed fashion. The program instructions may be executed by one or more processors. The computer-readable media may also be embodied in at least one application specific integrated circuit (ASIC) or Field Programmable Gate Array (FPGA), which executes (processes like a processor) program instructions. Examples of program instructions include both machine code, such as produced by a compiler, and files containing higher level code that may be executed by the computer using an interpreter. The above-described devices may be configured to act as one or more software modules in order to perform the operations of the above-described embodiments, or vice versa.

While the present disclosure has been particularly shown and described with reference to exemplary embodiments thereof, it will be understood by one of ordinary skill in the art that various changes in form and details may be made therein without departing from the spirit and scope of the present invention as defined by the following claims. The exemplary embodiments should be considered in descriptive sense only and not for purposes of limitation. Therefore, the scope of the present invention is defined not by the detailed description of the present disclosure but by the appended claims, and all differences within the scope will be construed as being included in the present disclosure.

What is claimed is:

1. A method of generating a temperature map showing a temperature change before and after an ultrasound wave for treatment is irradiated on a treatment part of a predetermined organ, the method comprising:
generating a plurality of reference frames indicating images of an observed part comprising the treatment part in the predetermined organ in a patient during a predetermined period related to a movement cycle of the predetermined organ from echo signals that are transduced from reflected waves of ultrasound waves for diagnosis irradiated on the observed part during the predetermined period;
generating a current frame indicating an image of the observed part at a time the ultrasound wave for treatment is irradiated on the treatment part from the echo signals that are transduced from the reflected waves of the ultrasound waves for diagnosis irradiated on the observed part;
selecting a comparison frame that is one of the plurality of reference frames based on a similarity between the reference frames and the current frame; and
generating the temperature map showing the temperature change in the observed part based on a difference between the comparison frame and the current frame.

2. The method of claim 1, wherein the selecting of the comparison frame comprises selecting a frame that is the most similar to the current frame from among the reference frames as the comparison frame.

3. The method of claim 2, wherein the selecting of the comparison frame comprises determining a frame that is the most similar to the current frame from among the reference frames based on a difference between pixel values of each of the reference frames and pixel values of the current frame and selecting the reference frame, which is determined as the most similar frame to the current frame, as the comparison frame.

4. The method of claim 1, wherein the predetermined period comprises a breathing cycle of the patient that corresponds to the movement cycle of the predetermined organ, and
the generating of the plurality of reference frames comprises generating the reference frames during the breathing cycle of the patient.

5. The method of claim 1, wherein the predetermined period is a pause period between a breathing motion in which the movement of the predetermined organ is relatively small in the movement cycle of the predetermined organ, and
the generating of the plurality of reference frames comprises generating the reference frames during the pause period between the breathing motion.

6. The method of claim 5, wherein the generating of the current frame comprises generating the current frame from the echo signals that are transduced from the reflected waves of the ultrasound waves for diagnosis irradiated on the observed part during the pause period between the breathing motion.

7. The method of claim 1, wherein the generating of the current frame comprises generating current frames indicating images of the predetermined organ from the echo signals that are transduced from the reflected waves of the ultrasound waves for diagnosis irradiated on a plurality of cross-sectional images forming the observed part, and
the generating of the temperature map comprises generating a three-dimensional (3D) temperature map by accumulating a plurality of temperature maps generated from the generated current frames.

8. The method of claim 1, wherein the selecting of the comparison frame comprises selecting candidate reference frames from among the plurality of reference frames by considering an estimated position of the observed part at a time corresponding to the movement cycle of the predetermined organ or a time the current frame is generated.

9. The method of claim 1, wherein each of the reference frames is obtained by replacing a reference frame generated at a time corresponding to a time the current frame is generated with the current frame by considering the movement cycle of the predetermined organ.

10. The method of claim 1, wherein each of the generating of the temperature map comprises generating the temperature map by detecting a different type of waveform change between echo signals for generating the comparison frame selected from among the reference frames and echo signals for generating the current frame.

11. A non-transitory computer-readable recording medium storing a computer-readable program to implement the method of claim 1.

12. An ultrasound system to generate a temperature map showing a temperature change before and after an ultrasound wave for treatment is irradiated on a treatment part of a predetermined organ in a patient, the ultrasound system comprising:
an ultrasound diagnosis device to irradiate ultrasound waves for diagnosis on an observed part comprising the treatment part in the predetermined organ inside the patient during a predetermined period related to a movement cycle of the predetermined organ;
an ultrasound treatment device to irradiate the ultrasound waves for treatment on the treatment part; and
an ultrasound data processing device to generate the temperature map showing the temperature change in the observed part based on a difference between any one of a plurality of reference frames indicating images of the observed part that are generated from echo signals transduced from reflected waves of the ultrasound waves for diagnosis irradiated during the predetermined period and a current frame indicating an image of the observed part that is generated at a time the ultrasound wave for treatment is irradiated on the treatment part from the echo signals that are transduced from the reflected waves of the ultrasound waves for diagnosis.

13. The ultrasound system of claim 12, wherein the ultrasound data processing device comprises a comparison frame generator for selecting a frame that is the most similar to the current frame from among the reference frames as a comparison frame.

14. The ultrasound system of claim 13, wherein the comparison frame generator determines a frame that is the most similar to the current frame from among the reference frames based on a difference between pixel values of each of the reference frames and pixel values of the current frame and selects the reference frame, which is determined as the most similar frame to the current frame, as the comparison frame.

15. The ultrasound system of claim 12, wherein the predetermined period is a breathing cycle of the patient that corresponds to the movement cycle of the predetermined organ, and
the ultrasound data processing device comprises a reference frame generator for generating the reference frames during the breathing cycle of the patient.

16. The ultrasound system of claim 12, wherein the predetermined period is a pause period between a breathing motion in which the movement of the predetermined organ is relatively small in the movement cycle of the predetermined organ, and
the ultrasound data processing device comprises a reference frame generator for generating the reference frames during the pause period between the breathing motion.

17. The ultrasound system of claim 16, wherein the reference frame generator generates the current frame from the echo signals that are transduced from the reflected waves of the ultrasound waves for diagnosis irradiated on the observed part during the pause period between the breathing motion.

18. The ultrasound system of claim 12, wherein the ultrasound data processing device comprises:
a current frame generator to generate current frames indicating images of the predetermined organ from the echo signals that are transduced from the reflected waves of the ultrasound waves for diagnosis irradiated on a plurality of cross-sectional images forming the observed part; and
a temperature map generator to generate a three-dimensional (3D) temperature map by accumulating a plurality of temperature maps generated from the generated current frames.

19. The ultrasound system of claim 15, wherein the reference frame generator further comprises a reference frame selector to select candidate reference frames from among the plurality of reference frames by considering an estimated position of the observed part at a time corresponding to the movement cycle of the predetermined organ or a time the current frame is generated.

20. The ultrasound system of claim 15, wherein the reference frame generator replaces a reference frame generated at a time corresponding to a time the current frame is generated with the current frame by considering the movement cycle of the predetermined organ.
